(19) United States
(12) Patent Application Publication
(10) Pub. No.: US 2026/0103683 A1
(43) Pub. Date: Apr. 16, 2026

(54) METHODS OF GENERATING BRAIN ORGANOIDS AND REPAIRING NON-VIABLE EMBRYOID BODIES

(71) Applicant: UNIVERSITY OF SASKATCHEWAN, Saskatoon (CA)

(72) Inventors: Tyler Joseph Richard WENZEL, Saskatoon (CA); Darrell MOUSSEAU, Saskatoon (CA)

(21) Appl. No.: 19/116,830

(22) PCT Filed: Sep. 27, 2023
(86) PCT No.: PCT/CA2023/051277

§ 371 Date: Mar. 28, 2025

Related U.S. Application Data
(60) Provisional application No. 63/410,876, filed on Sep. 28, 2022.
(60) Provisional application No. 63/530,557, filed on Aug. 3, 2023.

(51) Int. Cl. C12N 5/071; C12N 5/00; G01N 33/68

(52) U.S. Cl. CPC C12N 5/0697; C12N 5/0018; G01N 33/6896; C12N 2500/12; C12N 2500/25; C12N 2500/33; C12N 2500/38; C12N 2500/84; C12N 2501/115; C12N 2501/13; C12N 2501/15; C12N 2501/91; C12N 2501/998; C12N 2503/00; C12N 2506/45; C12N 2513/00; C12N 2523/00; G01N 2333/4703; G01N 2333/4709; G01N 2800/2821

(57) ABSTRACT

The disclosure relates to methods of generating at least one brain organoid-sufficient embryoid body or repairing at least one non-viable embryoid body, the method comprising incubating a population of induced pluripotent stem cells (iPSCs) in an embryoid body formation medium (EB FM), the EB FM comprising a culture medium, glutamine, recombinant human insulin, recombinant human transferrin, sodium selenite, and thermostable fibroblast growth factor 2 (FGF2). The disclosure further relates to methods of generating brain organoids from brain organoid-sufficient embryoid bodies and repairing embryoid bodies.

### CROSS-REFERENCE TO RELATED APPLICATIONS

This application claims the benefit of priority to U.S. Provisional Application No. 63/410,876, filed Sep. 28, 2022, and U.S. Provisional Application No. 63/530,557, filed Aug. 3, 2023, the contents of which are incorporated herein by reference in their entirety.

### INCORPORATION OF SEQUENCE LISTING

A computer readable form of the Sequence Listing “13764-P68588PC00_SequenceListing” (4,112 bytes) created on Sep. 21, 2023, is herein incorporated by reference.

### FIELD

The present disclosure relates to methods of generating brain organoids, in particular, methods of generating brain organoids from organoid-sufficient embryoid bodies and repaired embryoid bodies.

### BACKGROUND

Mechanisms involved in brain development and homeostasis are complex, and while considerable insight into the mechanisms driving these processes has been gathered from animal models, species-specific differences continue to challenge the application of knowledge derived from animals unequivocally to the human context. Organoids are a three-dimensional in vitro culture that involve the differentiation of stem cells into organ-specific cell types that subsequently self-organize into patterns that resemble those of the eponymous organ. While stem cells from several species can be used, the use of human stem cells generates an organoid that has the potential to replace animal models. The self-patterning seen with organoids recapitulates the cytoarchitecture and functions of the eponymous human organ and the presence of a human genome allows for more directed genetic manipulations.

Organoid technologies, including those described for brain organoids, are in their infancy. There is limited understanding about organoid formation using self-patterned protocols. Several such protocols exist for brain organoids. The protocol by Lancaster & Knoblich (2014) uses undefined components such as fetal bovine serum and KnockOut™ Serum Replacement, whereas commercial kits use a proprietary formulation that is marketed as serum-free. Such chemically undefined formulations complicate the study of brain organoid formation, as they do not allow full control over their development nor do they allow for sufficient troubleshooting, when necessary.

Brain organoids are more representative of the brain tissues since they are composed of several brain cell types self-arranged in a three-dimensional shape. Use of organoids instead of mono or mixed two-dimensional cultures of brain cells may also result in a more physiologically or pathologically relevant distribution pattern of internalized molecules, since it has been shown, for example, that the endocytic activity of astrocytes is dependent on the presence of other brain cell types (Konishi et al., 2020).

Additionally, healthy (i.e., homeostatic) microglia are characterized by low levels of IBA1 (ionized calcium-binding adapter molecule 1) and high levels of TMEM119 (transmembrane protein 119) (Lier et al., (2021)). In the brain, proteins IBA1 and TMEM119 are only expressed by microglia. IBA1 regulates actin dynamics that are critical for cell migration and phagocytosis by microglia (Ohsawa et al., (2004)), and although the function of TMEM119 is unclear, it may help with cell proliferation and migration by activating the Wnt/beta-catenin pathway (Yang et al., (2021)). Complicating the study of healthy microglial responses to endogenous molecules, microglia convert to a non-healthy (i.e., non-homeostatic) state when cultured in isolation in a dish (Gosselin et al., (2017)). Thus, isolated microglia cannot inform normal physiological responses to signalling molecules. Animal-derived microglia are equally limited in that they differ from human microglia in their expression of receptors, regulation of genes and the existence of signalling cascades (Yang et al., (2021); Gosselin et al., (2017)).

Microglia are the innate immune cells of the brain and help maintain homeostasis, regulate synaptic development as well as facilitate the maturation of other brain cell types. Brain organoid platforms are gaining widespread interest as models of the human brain and microglia will likely play a critical role in generating the most translationally relevant brain organoid platform. Current protocols generate brain organoids 1) that are devoid of microglia, or that incorporate microglia through either 2) co-culture or gene editing techniques, or 3) specific molecular cocktails. It is currently unclear whether these diverse protocols exert any overt effect on microglial function or physiology once they are incorporated into the brain organoid.

### SUMMARY

Herein is described a self-patterned whole-brain organoid protocol that gives rise to in vitro cultures comprised of astrocytes, neurons, oligodendrocytes, and microglia after only ninety days in culture. Astrocytes and oligodendrocytes do not often arise until 180 days in brain organoid cultures, while current protocols commonly exclude microglia (Tanaka et al. 2020; Sivitilli et al. 2020). With the present induced pluripotent stem cell (iPSC) lines, the protocol described herein produced highly viable and uniform organoids composed of astrocytes, neurons, oligodendrocytes, and microglia and at yields greatly exceeding those of commercial kits, the protocol described in Lancaster & Knoblich (2014), and other organ-specific protocols equally lacking in nutrients critical for the formation of healthy embryoid bodies (EBs). It is also demonstrated that the unique media formulations described herein repair malformed EBs as they overcome nutrient deficits of other formulations. The formation of EBs is the initial stage of brain organoid generation and their successful formation is critical to any protocol. Inadequately formed EBs are often misattributed to substandard iPSCs (Lancaster & Knoblich, 2014); however, it is now demonstrated that inadequate EBs generated from the same culture source of iPSCs can be repaired within 24 hours of incubation in the newly formulated medium described herein and can be differentiated further into ‘healthy’ brain organoids.

Accordingly, a first aspect of the disclosure includes a method of generating at least one brain organoid-sufficient embryoid body, the method comprising incubating a population of induced pluripotent stem cells (iPSCs) in an embryoid body formation medium (EB FM), the EB FM comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin;
about 5 ng/ml to about 30 ng/ml of sodium selenite; and
about 1 ng/ml to about 200 ng/ml, optionally about 10 ng/ml to about 25 ng/ml of thermostable fibroblast growth factor 2 (FGF2).

Another aspect of the disclosure includes a method of repairing a non-viable embryoid body, the method comprising
obtaining a population of embryoid bodies (EBs) comprising at least one non-viable embryoid body, and
incubating the population of embryoid bodies with an embryoid body formation medium (EB) FM, wherein the EB FM comprises:
   cell culture medium, optionally Dulbecco's modified Eagle medium (DMEM);
   about 1 μM to about 100 mM glutamine, optionally about 0.1 mM to about 10 mM L-alanyl-L-glutamine dipeptide;
   about 5 μg/ml to about 30 μg/ml of recombinant human insulin;
   about 5 μg/ml to about 30 μg/ml of recombinant human transferrin; and
   about 5 ng/ml to about 30 ng/ml of sodium selenite.

Another aspect of the disclosure includes a method of generating at least one brain organoid, the method comprising
a. generating at least one brain organoid-sufficient embryoid body using any method described herein and/or generating at least one repaired brain organoid-sufficient embryoid body using any method described herein;
b. transferring the at least one brain organoid-sufficient embryoid body generated in step a) from the EB FM used in step a) to a neural induction medium without transferring any of the EB FM into the neural induction medium and incubating the at least one brain organoid-sufficient embryoid body in the neural induction medium for at least about 24 hours and up to about 96 hours, optionally for about 48 hours;
c. replacing the neural induction medium with expansion medium having a temperature of at least about 0° C. and up to about 12° C., optionally at least about 4° C. to about 6° C.; and
d. replacing the expansion medium with maturation medium after at least about 48 hours up to about 96 hours, optionally at least about 72 hours.

Another aspect of the disclosure includes an embryoid body formation medium (EB FM) for generating at least one brain organoid-sufficient embryoid body, the EB FM comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin;
about 5 ng/ml to about 30 ng/ml of sodium selenite; and
about 1 ng/ml to about 200 ng/ml, optionally about 10 ng/ml to about 25 ng/ml of thermostable fibroblast growth factor 2 (FGF2).

Another aspect of the disclosure includes an embryoid body formation medium (EB FM) for repairing at least one non-viable embryoid body, the EB FM comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin; and
about 5 ng/ml to about 30 ng/ml of sodium selenite.

Another aspect of the disclosure includes brain organoids produced by the processes described herein, brain organoid-sufficient embryoid bodies produced by the processes described herein, and/or brain organoid-sufficient embryoid bodies repaired by the processes described herein.

Another aspect of the disclosure includes a method of diagnosing or predicting onset of Alzheimer's disease in a patient suspected of having or developing Alzheimer's disease, the method comprising:
a) collecting a sample from the patient,
b) isolating cells from the sample,
c) reprogramming the cells into induced pluripotent stem cells (iPSCs),
d) using the iPSCs to generate a brain organoid using any of the methods described herein, and
e) detecting the presence of markers associated with Alzheimer's disease in the brain organoid.

### BRIEF DESCRIPTION OF THE DRAWINGS

Embodiments of the present disclosure will now be described in relation to the drawings in which:

FIGS. 1A-C: Representative images of EB formation data in Tables 6-7. iPSCs from the same cell suspension were seeded in either (FIG. 1A, top) embryoid body formation medium (EB FM) 1, (bottom) EB FM 4-6, or (FIG. 1B) EB FM 2, which is the most cited EB FM in the literature. (FIG. 1A,top) After 24 h, EB FM 1 always formed multiple, small, inadequate EBs for brain organoid generation, whereas (bottom) EB FM 4-6 formed the single, viable EB required for brain organoid generation. (FIG. 1B) After 24 h, EB FM 2 produced an aggregate with undefined borders, which disaggregated into a monolayer of single cells by 48 h. (FIG. 1C) Inadequate EBs formed by EB FM 1 were repaired within 24 hours post-exposure to EB FM 4 and 6. Scale bars: 100 μm.

FIGS. 2A-D: EB FM used dictates the cellular composition of brain organoids. (FIG. 2A) Viable EBs successfully develop into brain organoids. By day 6, all viable EBs exhibit the characteristic brightening of their outer edge indicating the formation of the neuroectoderm. On day 9, all viable EBs display neuroepithelial budding and by day 16 the neuroepithelium visibly grew. Day 90 is a representative image of a mature brain organoid. (FIG. 2B) Brain organoids grown from different EB FMs were cryosectioned and glial markers immunostained. (FIG. 2C) Protein lysate from a subset of brain organoids were used in immunoblotting to measure the immunodensities of GFAP, TMEM119, TUBB3, OLIG2, and β-actin. * p<0.05, ** p<0.01 according to the unpaired Mann-Whitney test. Representative bands are shown. (FIG. 2D) Brain organoids grew to a comparable size regardless of EB FM used (p>0.1) according to the unpaired Kruskal-Wallis and Dunn's post hoc tests. Data presented as mean±SD.

FIGS. 3A-C: The presence of astrocytes and microglia was associated with higher levels of synaptic proteins. Protein lysate from the same subset of brain organoids in FIG. 2 were used to immunoblot for (FIG. 3A) CX43, (FIG. 3B) SYN1, (FIG. 3C) PSD95. * p<0.05 according to the unpaired Mann-Whitney test. Representative bands are shown. Data presented as mean±SD.

FIGS. 4A-B: iPSC-derived microglia were positive for microglial markers (TMEM19, IBA1) and negative for the neuronal lineage marker TUBB3 (FIG. 4A). Brain organoids were positive for markers of oligodendroglial (OLIG2), astrocytic (GFAP), microglial (IBA1, TMEM119), and neuronal (TUBB3) lineages (FIG. 4B).

FIGS. 5A-H: BOs were grown as described herein, and (FIG. 5A) these BOs, regardless of cell line used, exhibit all visual markers of proper development. (FIG. 5B) Immunoblotting analysis indicates that BOs become more complex as they are cultured for longer periods of time, as exemplified by the presence and maturation of GFAP and SYN1. At day 90, (FIGS. 5C-F) the levels of housekeeping proteins TUBB3, β-actin, and GAPDH are consistently expressed at similar levels, (FIG. 5G) as are the levels of total protein, indicating these cultures grow to similar sizes and complexities at similar rates. (FIG. 5H) The ability of BOs to metabolize resazurin into resorufin is also similar at day 90, indicating similar metabolic activity. (FIGS. 5D-H) Data (means±SD) analyzed according to Mann-Whitney test. (FIGS. 5B-F) BO data were derived by pooling five organoids. The five pooled BOs were from the same batch, but each sample (i.e., lane or datapoint) was from a different batch of five organoids (batches defined as BOs generated on different days from iPSCs of a different passage number). (FIGS. 5G,H) Total protein and resorufin datapoints represent individual organoids.

FIGS. 6A-D: Summary of immunoblotting densitometry comparing the expression levels of (FIG. 6A) neuron-associated markers (i, NeuN 55 kDa; ii, NeuN 110 kDa; iii, TUBB3; iv, SYN1), (FIG. 6B) microglia-associated markers (i, IBA1; ii, TMEM119; iii, P2RY12 25 kDa; iv, P2RY12 60 kDa), (FIG. 6C) macroglia (astrocyte and oligodendrocyte)-associated markers (i, GFAP; ii, OLIG2), and (FIG. 6D) other proteins of interest (i, TLR4; ii, β-actin). Data presented as means±SD and normalized to the housekeeping protein GAPDH. Datapoints from primary mouse and human tissues were derived from different donors, while BO datapoints were derived by pooling five organoids. The five BOs were from the same batch, but each sample (i.e., datapoint) was from a different batch of five organoids (batches defined as BOs generated on different days from iPSCs of a different passage number). * P<0.05, ** P<0.01, and *** P<0001 according to the Dunn's post hoc test. Only human cerebellar tissue exhibited sex differences in protein levels (data not shown).

FIGS. 7A-D: Immunoblotting results demonstrating banding patterns in female and male human cortex, human cerebellum, human brain organoid, C57BI/6J mouse cortex for NeuN, TUBB3, SYN1 (FIG. 7A), IBA1, TMEM119, P2RY12 (FIG. 7B), GFAP, OLIG2 (FIG. 7C), TLR4, beta-actin, GAPDH (FIG. 7D). NeuN (neuronal nuclei marker) and TMEM119 (microglia marker) exhibit banding patterns in a species-specific manner. The characteristic doublet for NeuN is roughly 90 kDa in primary human brain and BO tissues, but the doublet resolves between 55-70 kDa in mouse cortical tissues. There is a protein detected between 55-70 kDa in human tissues, but it is not a doublet. TMEM119 is often described as a 55 kDa protein, and a protein band clearly appears at this molecular weight in mouse cortical tissues. However, a triplet between 40-55 kDa is detected in both primary human brain and BO tissues. SYN1 is often described as a 77 kDa protein, and sometimes antibodies detect a doublet that corresponds with SYN1a and SYN1b. In the human tissues (human brain and brain organoid), there is a protein doublet detected between 70-100 kDa, but the protein doublet resolves between 55-70 kDa in mouse cortical tissue. GFAP is often described as a 55 kDa protein but has several known isoforms that are between 35-55 kDa. Several GFAP isoforms were detected in human brain and brain organoids, whereas it appears that mouse cortical tissues have predominately one isoform of GFAP and it is expressed at lower levels. The P2RY12 antibodies detect protein at 17 kDa and around 40 kDa in human tissues (human brain and brain organoids), whereas only a stronger 40 kDa band was detected in mouse cortical tissues. B-actin, IBA1, GAPDH OLIG2, TLR4, and TUBB3 exhibited similar protein banding patterns between all tissues tested. The 17 kDa band that appears in TMEM119 and OLIG2 blots was noted, but this unexpected band was disregarded as it could be an aggregate of breakdown products. Regardless, protein banding around this molecular weight was observed in all tissues. Datapoints from primary mouse and human tissues were derived from different donors, while BO datapoints were derived by pooling five organoids. The five BOs were from the same batch, but each sample (i.e., lane) was from a different batch of five organoids (batches defined as BOs generated on different days from iPSCs of a different passage number).

FIGS. 8A-F are a series of images which depict protein banding patterns and graphs depicting immunodensity of amyloid-beta proteins in brain organoids after the addition of exogenous amyloid-beta to the culture medium for 24 hours. Brain organoids were generated using the methods described herein (FIGS. 8A, C, E and F) or other methods (i.e., co-culturing) (FIGS. 8B, D, E and F). FIG. 8F, top image represents Co-culture Batch 1, FIG. 8F, middle image represents Co-culture Batch 2, and FIG. 8F, bottom image represents Innage Batch 1.

FIGS. 9A-E are a series of graphs depicting immunodensity of IBA1 (FIGS. 9A, B, D), GFAP (FIGS. 9A, C, E), and GAPDH (FIG. 9A), proteins in human cortical tissues from female and male donors with familial Alzheimer's disease, FAD; or late-onset Alzheimer's disease, LOAD; or control tissue.

FIGS. 10A-C are a series of graphs (FIGS. 10B-C) depicting immunodensity of IBA1, TUBB3, and GAPDH proteins and images (FIG. 10A) which depict protein banding patterns at different time points in brain organoids generated using the methods described herein.

FIGS. 11A-C are a series of images depicting protein banding patterns of IBA1, phosphorylated tau, SYN1, GFAP, beta-actin (FIG. 11A), and amyloid beta peptides in brain organoids generated from iPSCs from a female Alzheimer's disease patient (AD (female) BO) and a healthy female (87i (female) BO) (FIG. 11B) using the methods described herein, as well as change in colour in media from organoids derived from Alzheimer's patients (right panel of FIG. 11C) as compared to media from organoids derived from healthy humans (left two panels of FIG. 11C).

FIGS. 12A-C: Two weeks after attaching the BOs to the multielectrode array, the multielectrode array detects neural network activity using multielectrode arrays in 86i (male) BOs 90 days after initial embryoid body formation. FIG. 12A depicts observed spontaneous actional potentials (spikes) and network burst electrical activity. FIGS. 12B-C demonstrate the electrophysiological properties of the network activity.

### DETAILED DESCRIPTION

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. For example, the term “a cell” includes a single cell as well as a plurality or population of cells. Generally, nomenclatures utilized in connection with, and techniques of, cell and tissue culture, molecular biology, and protein and oligonucleotide or polynucleotide chemistry and hybridization described herein are those well-known and commonly used in the art (see, e.g., Green, M. and Sambrook, J. (2012) Molecular Cloning: A Laboratory Manual. 4th Edition, Vol. II, Cold Spring Harbor Laboratory Press, New York.).

As used in this specification and the appended claims, the singular forms “a”, “an” and “the” include plural references unless the content clearly dictates otherwise. Thus, for example, a composition containing “a compound” includes a mixture of two or more compounds. It should also be noted that the term “or” is generally employed in its sense including “and/or” unless the content clearly dictates otherwise.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

In understanding the scope of the present disclosure, the term “comprising” and its derivatives, (such as “comprise” and “comprises”), “having” (and any form of having, such as “have” and “has”), “including” (and any form of including, such as “include” and “includes”) or “containing” (and any form of containing, such as “contain” and “contains”), as used herein, are intended to be open ended terms that specify the presence of the stated features, elements, components, groups, integers, and/or steps, but do not exclude the presence of other unstated features, elements, components, groups, integers and/or steps.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

As used herein, the term “brain organoid” refers to a three-dimensional in vitro culture derived from stem cells into brain-specific cell types that subsequently self-organize into patterns that resemble those of the brain.

As used herein, the term “brain organoid-sufficient embryoid body” refers to an embryoid body which is capable of generating a brain organoid, for example when exposed to neural induction media, expansion media, and maturation media, for example as in a method described herein. A brain organoid-sufficient embryoid body includes an embryoid body comprising a sphere of aggregated cells with a smooth border as compared to a jagged border.

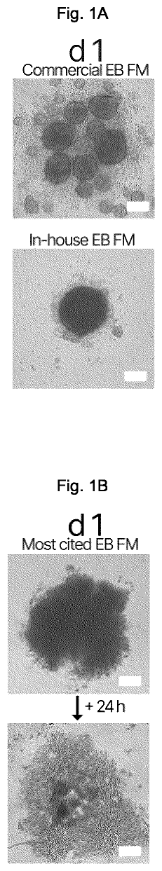

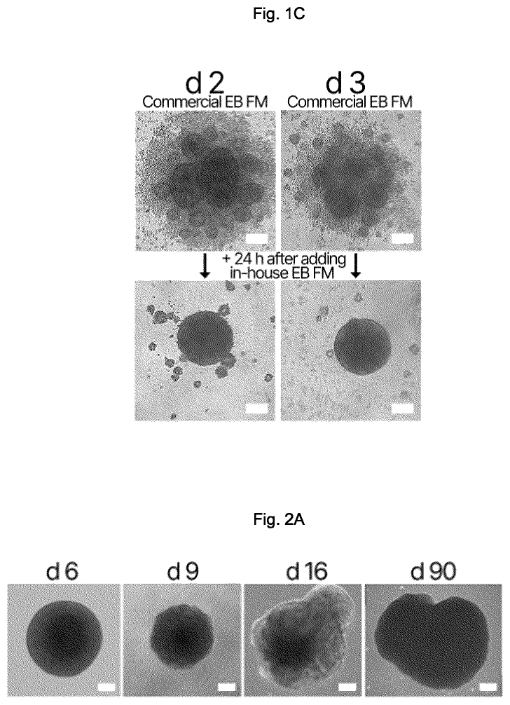

As used herein, the term “non-viable embryoid body” refers to a malformed embryoid body comprising at least one living cell, for example those that do not exhibit smooth borders, but rather a jagged border, do not aggregate into a sphere, or aggregate into multiple spheres and/or will not generate a brain organoid when exposed to the methods described herein for generating brain organoids. Examples of non-viable embryoid bodies are shown in for example, FIG. 1A, top image and FIG. 1B.

As used herein, the term “repaired embryoid body” or related terms refers to an embryoid body which was malformed but has been restored to a viable state for example to a brain organoid-sufficient embryoid body, for example using the methods described herein.

As used herein, the term “cell culture medium” refers to a medium that can support cell life, examples of which are known in the art.

As used herein, the term “neural induction medium” refers to a medium for the neural induction of human embryonic stem cells and induced pluripotent stem cells and examples of such media are known in the art.

As used herein, the term “expansion medium” refers to a chemically defined medium useful for expansion of embryoid bodies and examples of such media are known in the art, minimally comprises an extracellular matrix such as Geltrex, Matrigel, and VitroGel, and a cell culture medium, and lacks vitamin A.

As used herein, the term “maturation medium” refers to a chemically defined medium useful for development of brain organoids from embryoid bodies and examples of such medium are well known in the art and minimally comprises an extracellular matrix such as Geltrex, Matrigel, and VitroGel, a cell culture medium, and vitamin A.

As used herein, the term “thermostable FGF2” refers to a genetically modified fibroblast growth factor 2 (FGF2) peptide comprising mutations that render it thermally stable, examples of which are known in the art. Thermally stable genetically modified FGF2 peptide include peptides comprising, for example, the amino acid sequence MAAGSITTLP ALPEDGGSGA FPPGHFKDPK RLYCKNGGFF LRIHPDGRVD GVREKSDPHI KLQLQAEERG VVSIKGVCAN RYLAMKEDGR LLASKCVTDE CFFFERLESN NYNTYRSRKY TSWYVALKRT GQYKLGSKTG PGQKAILFLP MSAKS (SEQ ID NO: 3) comprising one or more mutations at residue 31, 52, 54, 59, 92, 94, 96, 109, 121, and/or 128 of SEQ ID NO: 3 or conservatively substituted variants thereof. Specific examples of thermally stable genetically modified FGF2 peptide include peptides comprising an amino acid sequence such as:

MAAGSITTLP ALPEDGGSGA FPPGHFKDPK RLYCKNGGFF LRIHPDGRVD GVREKSDPHI KLQLQAEERG VVSIKGVCAN RYLAMKEDGR LLASKCVTDE CFFFERLESN NYNTYRSRKY TSWYVALNRT GQYKLGSKTG PGQKAILFLP MSAKS (SEQ ID NO: 1) or MAAGSITTLP ALPEDGGSGA FPPGHFKDPK LLYCKNGGFF LRIHPDGRVD GTRDKSDPFI KLQLQAEERG VVSIKGVCAN RYLAMKEDGR LYAIKNVTDE CFFFERLEEN NYNTYRSRKY PSWYVALKRT GQYKLGSKTG PGQKAILFLP MSAKS (SEQ ID NO: 2) or conservatively substituted variants thereof. The methods described herein have been demonstrated with different thermostable FGF2, including SEQ ID NO: 1 and SEQ ID NO: 2, and their use results in yields similar to the experimental data reported herein.

As used herein, the term “Dulbecco's modified Eagle medium (DMEM)” refers to a composition comprising the ingredients in Table 1.

TABLE 1
Components of DMEM

| | | | |
|---|---|---|---|
| | Molecular | Concentration | |
| Components | Weight | (mg/L) | mM |

Amino Acids

| | | | |
|---|---|---|---|
| Glycine | 75.0 | 30.0 | 0.4 |
| L-Arginine hydrochloride | 211.0 | 84.0 | 0.39810428 |
| L-Cystine 2HCl | 313.0 | 63.0 | 0.20127796 |
| L-Glutamine | 146.0 | 584.0 | 4.0 |
| L-Histidine hydrochloride- | 210.0 | 42.0 | 0.2 |
| H2O | | | |
| L-Isoleucine | 131.0 | 105.0 | 0.8015267 |
| L-Leucine | 131.0 | 105.0 | 0.8015267 |
| L-Lysine hydrochloride | 183.0 | 146.0 | 0.7978142 |
| L-Methionine | 149.0 | 30.0 | 0.20134228 |
| L-Phenylalanine | 165.0 | 66.0 | 0.4 |
| L-Serine | 105.0 | 42.0 | 0.4 |
| L-Threonine | 119.0 | 95.0 | 0.79831934 |
| L-Tryptophan | 204.0 | 16.0 | 0.078431375 |
| L-Tyrosine disodium salt | 261.0 | 104.0 | 0.39846742 |
| dihydrate | | | |
| L-Valine | 117.0 | 94.0 | 0.8034188 |

Vitamins

| | | | |
|---|---|---|---|
| Choline chloride | 140.0 | 4.0 | 0.028571429 |
| D-Calcium pantothenate | 477.0 | 4.0 | 0.008385744 |
| Folic Acid | 441.0 | 4.0 | 0.009070295 |
| Niacinamide | 122.0 | 4.0 | 0.032786883 |
| Pyridoxine hydrochloride | 206.0 | 4.0 | 0.019417476 |
| Riboflavin | 376.0 | 0.4 | 0.0010638298 |
| Thiamine hydrochloride | 337.0 | 4.0 | 0.011869436 |
| i-Inositol | 180.0 | 7.2 | 0.04 |

Inorganic Salts

| | | | |
|---|---|---|---|
| Calcium Chloride (CaCl2) | 111.0 | 200.0 | 1.8018018 |
| (anhyd.) | | | |
| Ferric Nitrate | 404.0 | 0.1 | 2.4752476E−4 |
| (Fe(NO3)3″9H2O) | | | |
| Magnesium Sulfate | 120.0 | 97.67 | 0.8139166 |
| (MgSO4) (anhyd.) | | | |
| Potassium Chloride (KCl) | 75.0 | 400.0 | 5.3333335 |
| Sodium Bicarbonate | 84.0 | 3700.0 | 44.04762 |
| (NaHCO3) | | | |
| Sodium Chloride (NaCl) | 58.0 | 6400.0 | 110.344826 |
| Sodium Phosphate | 138.0 | 125.0 | 0.9057971 |
| monobasic | | | |
| (NaH2PO4—H2O) | | | |

Other Components

| | | | |
|---|---|---|---|
| D-Glucose (Dextrose) | 180.0 | 4500.0 | 25.0 |
| Phenol Red | 376.4 | 15.0 | 0.039851222 |

As used herein, the term “Dulbecco's modified Eagle medium with F-12 Ham nutrient mixture (DMEM-F12) with 4-(2-hydroxyethyl)-1-piperazineethanesulfonic acid (HEPES)” refers to a composition comprising the ingredients in Table 2.

TABLE 2
Components of DMEM F12 with HEPES

| | | | |
|---|---|---|---|
| | Molecular | Concentration | |
| Components | Weight | (mg/L) | mM |

Amino Acids

| | | | |
|---|---|---|---|
| Glycine | 75.0 | 18.75 | 0.25 |
| L-Alanine | 89.0 | 4.45 | 0.049999997 |
| L-Arginine hydrochloride | 211.0 | 147.5 | 0.69905216 |
| L-Asparagine-H2O | 150.0 | 7.5 | 0.05 |
| L-Aspartic acid | 133.0 | 6.65 | 0.05 |
| L-Cysteine hydrochloride-H2O | 176.0 | 17.56 | 0.09977272 |
| L-Cystine 2HCI | 313.0 | 31.29 | 0.09996805 |
| L-Glutamic Acid | 147.0 | 7.35 | 0.05 |
| L-Glutamine | 146.0 | 365.0 | 2.5 |
| L-Histidine hydrochloride-H2O | 210.0 | 31.48 | 0.14990476 |
| L-Isoleucine | 131.0 | 54.47 | 0.41580153 |
| L-Leucine | 131.0 | 59.05 | 0.45076334 |
| L-Lysine hydrochloride | 183.0 | 91.25 | 0.4986339 |
| L-Methionine | 149.0 | 17.24 | 0.11570469 |
| L-Phenylalanine | 165.0 | 35.48 | 0.2150303 |
| L-Proline | 115.0 | 17.25 | 0.15 |
| L-Serine | 105.0 | 26.25 | 0.25 |
| L-Threonine | 119.0 | 53.45 | 0.44915968 |
| L-Tryptophan | 204.0 | 9.02 | 0.04421569 |
| L-Tyrosine disodium salt dihydrate | 261.0 | 55.79 | 0.21375479 |
| L-Valine | 117.0 | 52.85 | 0.4517094 |

Vitamins

| | | | |
|---|---|---|---|
| Biotin | 244.0 | 0.0035 | 1.4344263E−5 |
| Choline chloride | 140.0 | 8.98 | 0.06414285 |
| D-Calcium pantothenate | 477.0 | 2.24 | 0.0046960167 |
| Folic Acid | 441.0 | 2.65 | 0.0060090707 |
| Niacinamide | 122.0 | 2.02 | 0.016557377 |
| Pyridoxine hydrochloride | 206.0 | 2.0 | 0.009708738 |
| Riboflavin | 376.0 | 0.219 | 5.824468E−4 |
| Thiamine hydrochloride | 337.0 | 2.17 | 0.0064391694 |
| Vitamin B12 | 1355.0 | 0.68 | 5.0184503E−4 |
| i-Inositol | 180.0 | 12.6 | 0.07 |

Inorganic Salts

| | | | |
|---|---|---|---|
| Calcium Chloride (CaCl2) (anhyd.) | 111.0 | 116.6 | 1.0504504 |
| Cupric sulfate (CuSO4—5H2O) | 250.0 | 0.0013 | 5.2E−6 |
| Ferric Nitrate (Fe(NO3)3″9H2O) | 404.0 | 0.05 | 1.2376238E−4 |
| Ferrous Sulfate (FeSO4 7H2O) | 278.0 | 0.417 | 0.0015 |
| Magnesium Chloride (anhydrous) | 95.0 | 28.64 | 0.30147368 |
| Magnesium Sulfate (MgSO4) (anhyd.) | 120.0 | 48.84 | 0.407 |
| Potassium Chloride (KCl) | 75.0 | 311.8 | 4.1573334 |
| Sodium Bicarbonate (NaHCO3) | 84.0 | 1200.0 | 14.285714 |
| Sodium Chloride (NaCl) | 58.0 | 6995.5 | 120.61207 |
| Sodium Phosphate dibasic | 142.0 | 71.02 | 0.50014085 |
| (Na2HPO4) anhydrous | | | |
| Sodium Phosphate monobasic | 138.0 | 62.5 | 0.45289856 |
| (NaH2PO4-H2O) | | | |
| Zinc sulfate (ZnSO4—7H2O) | 288.0 | 0.432 | 0.0015 |

Other Components

| | | | |
|---|---|---|---|
| D-Glucose (Dextrose) | 180.0 | 3151.0 | 17.505556 |
| HEPES | 238.0 | 3574.5 | 15.018908 |
| Hypoxanthine Na | 159.0 | 2.39 | 0.015031448 |
| Linoleic Acid | 280.0 | 0.042 | 1.4999999E−4 |
| Lipoic Acid | 206.0 | 0.105 | 5.097087E−4 |
| Phenol Red | 376.4 | 8.1 | 0.021519661 |
| Putrescine 2HCl | 161.0 | 0.081 | 5.031056E−4 |
| Sodium Pyruvate | 110.0 | 55.0 | 0.5 |
| Thymidine | 242.0 | 0.365 | 0.0015082645 |

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### Methods

Accordingly, a first aspect of the disclosure includes a method of generating at least one brain organoid-sufficient embryoid body, the method comprising incubating a population of induced pluripotent stem cells (iPSCs) in an embryoid body formation medium (EB FM), the EB FM comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin;
about 5 ng/ml to about 30 ng/ml of sodium selenite; and
about 1 ng/ml to about 200 ng/ml, optionally about 10 ng/ml to about 25 ng/ml of thermostable fibroblast growth factor 2 (FGF2).

EB FMs consisting of the components described above were tested and resulted in average EB yields at about 75%.

In some embodiments, the EB FM further comprises:
about 1 μg/ml to about 1 mg/ml L-ascorbic acid (LAA);
about 5 mM to about 45 mM of sodium bicarbonate;
about 0.1 ng/ml to about 20 ng/ml of transforming growth factor beta-3 (TGF-β3) or about 1 ng/ml to about 200 ng/ml transforming growth factor beta-1 (TGF-β1); and/or about 0.1 ng/ml to about 100 ng/ml neuregulin 1 (NRG1).

EB FMs comprising these additional components were tested and have average EB yields at about 90%.

Concentration ranges of components, including those that are optional, comprised in the EB FMs described herein have been tested and their use results in yields similar to the experimental data included herein. Specifically, the concentration ranges of components described herein were tested and increased the yield of EBs to a minimum of 70%.

In some embodiments, the concentration of the glutamine in the EB FM is about 0.1 mM to about 10 mM. In some embodiments, the concentration of the glutamine in the EB FM is about 2 mM. In some embodiments, the concentration of the recombinant human insulin in the EB FM is about 20 μg/mL. In some embodiments, the concentration of the recombinant human transferrin in the EB FM is about 20 μg/ml. In some embodiments, the concentration of the sodium selenite in the EB FM is about 20 ng/ml. In some embodiments, the concentration of the LAA in the EB FM is about 0.2 mg/ml. In some embodiments, the concentration of the sodium bicarbonate in the EB FM is about 14.7 mM. In some embodiments, the concentration of the TGF-β3 in the EB FM is about 0.1 ng/ml. In some embodiments, the concentration of the NRG1 in the EB FM is about 0.1 ng/ml. In some embodiments, the concentration of the thermostable FGF2 in the EB FM is about 4 ng/ml. In some embodiments, the concentration of the thermostable FGF2 in the EB FM is about 4 ng/ml to about 50 ng/ml. In some embodiments, the concentration of the thermostable FGF2 in the EB FM is about 10 ng/ml to about 25 ng/ml. In some embodiments, the concentration of the thermostable FGF2 is about 50 ng/ml.

The glutamine in the EB FM may be present in the EB FM in the form of any compound that is a source of glutamine, including for example L-alanyl-L-glutamine dipeptide.

In some embodiments, the cell culture medium minimally comprises DMEM. In some embodiments, the cell culture medium is or comprises DMEM, DMEM with F-12 Ham nutrient mixture (DMEM-F12), or DMEM-F12 with 4-(2-hydroxyethyl)-1-piperazineethanesulfonic acid (HEPES).

The method reported herein would be expected to generate similar results with all types of pluripotent stem cells, including embryonic stem cells (ESCs), as those skilled in the art understand that the nutrient requirements of pluripotent stem cells are similar, and these cells have similar responses to molecules. In some embodiments, the population of iPSCs is from a patient having or suspected of having Alzheimer's disease. Alzheimer's disease includes for example familial Alzheimer's disease, sporadic Alzheimer's disease, early-onset Alzheimer's disease and late-onset Alzheimer's disease. In some embodiments, the Alzheimer's disease comprises familial Alzheimer's disease, sporadic Alzheimer's disease, early-onset Alzheimer's disease and/or late-onset Alzheimer's disease. In some embodiments, the Alzheimer's disease is sporadic Alzheimer's disease.

In some embodiments, the population of iPSCs is incubated in the EB FM for at least about 24 hours. In some embodiments, the population of iPSCs is incubated in the EB FM for at least about 24 hours and up to about 144 hours. In some embodiments, the population of iPSCs is incubated for about 24 hours, for about 48 hours, for about 72 hours, for about 96 hours, or about 144 hours. In some embodiments, the population of iPSC is incubated in the EB FM for at least about 72 hours. At about 96 hours, at least one brain organoid-sufficient embryoid body may be easily seen by the human eye, which may make it easier to move the at least one brain organoid-sufficient embryoid body. Accordingly, in some embodiments, the population of iPSCs is incubated for at least about 96 hours or at a time when the one brain organoid-sufficient embryoid body will be easily seen by the human eye.

In some embodiments, the method further comprises incubating the iPSCs in about 0.05 mM to about 5 mM ethylenediaminetetraacetic acid (EDTA) prior to incubation with the EB FM. Incubation time may depend on the cell line and concentration of EDTA used. In some embodiments, the concentration of EDTA is 0.5 mM. In some embodiments, the iPSCs are incubated in EDTA for between about 2 minutes and about 10 minutes. In some embodiments, the iPSCs are incubated in EDTA for about 4 minutes.

In some embodiments, the method further comprises adding further EB FM to the population of iPSCs after 24 hours of incubation. In some embodiments, the method further comprises adding further EB FM to the population of iPSCs after 72 hours of incubation.

Another aspect of the disclosure includes a method of repairing a non-viable embryoid body, the method comprising
obtaining a population of embryoid bodies (EBs) comprising at least one non-viable embryoid body, and
incubating the population of embryoid bodies with an embryoid body formation medium (EB) FM, wherein the EB FM comprises:
   a cell culture medium, optionally Dulbecco's modified Eagle medium (DMEM);
   about 1 μM to about 100 mM glutamine, optionally about 0.1 mM to about 10 mM L-alanyl-L-glutamine dipeptide;
   about 5 μg/ml to about 30 μg/ml of recombinant human insulin;
   about 5 μg/ml to about 30 μg/ml of recombinant human transferrin; and
   about 5 ng/ml to about 30 ng/ml of sodium selenite.

In some embodiments, the EB FM further comprises:
about 1 μg/ml to about 1 mg/ml L-ascorbic acid (LAA);
about 5 mM to about 45 mM of sodium bicarbonate;
about 0.1 ng/ml to about 20 ng/ml of transforming growth factor beta-3 (TGF-β3) or about 1 ng/ml to about 200 ng/ml transforming growth factor beta-1 (TGF-β1); and/or
about 0.1 ng/ml to about 100 ng/ml neuregulin 1 (NRG1).

In some embodiments, the EB FM further comprises about 1 ng/ml to about 200 ng/ml of thermostable FGF2. In some embodiments, the EB FM comprises about 10 ng/ml to about 25 ng/ml of thermostable FGF2. In some embodiments, the EB FM comprises about 4 ng/ml to about 50 ng/ml of thermostable FGF2. In some embodiments, the concentration of the thermostable FGF2 in the EB FM is about 4 ng/ml. In some embodiments, the concentration of the thermostable FGF2 in the EB FM is about 50 ng/ml.

In some embodiments, the concentration of the glutamine in the EB FM is about 0.1 mM to about 10 mM. In some embodiments, the concentration of the glutamine in the EB FM is about 2 mM. In some embodiments, the concentration of the recombinant human insulin in the EB FM is about 20 μg/mL. In some embodiments, the concentration of the recombinant human transferrin in the EB FM is about 20 μg/ml. In some embodiments, the concentration of the sodium selenite in the EB FM is about 20 ng/ml. In some embodiments, the concentration of the LAA in the EB FM is about 0.2 mg/ml. In some embodiments, the concentration of the sodium bicarbonate in the EB FM is about 14.7 mM. In some embodiments, the concentration of the TGF-β3 in the EB FM is about 0.1 ng/ml. In some embodiments, the concentration of the NRG1 in the EB FM is about 0.1 ng/ml.

The glutamine in the EB FM may be present in the EB FM in the form of any compound that is a source of glutamine, including for example L-alanyl-L-glutamine dipeptide.

In some embodiments, the cell culture medium minimally comprises DMEM. In some embodiments, the cell culture medium is or comprises DMEM, DMEM with F-12 Ham nutrient mixture (DMEM-F12), or DMEM-F12 with 4-(2-hydroxyethyl)-1-piperazineethanesulfonic acid (HEPES)

The amount of time a population of embryoid bodies is incubated in the EB FM is dependent on how many living cells the at least one non-viable organoid comprises at the time the EB FM is added. In some embodiments, the population of embryoid bodies is incubated in the EB FM for at least about 24 hours. In some embodiments, the population of embryoid bodies is incubated in the EB FM for between about 24 hours and about 144 hours. In some embodiments, the population of embryoid bodies is incubated in the EB FM for at least about 24 hours and up to about 72 hours. In some embodiments, the population of embryoid bodies is incubated in the EB FM for about 24 hours, for about 48 hours, for about 72 hours, or for about 144 hours. In some embodiments, the population of embryoid bodies is incubated in the EB FM for about 72 hours.

In some embodiments, the method further comprises adding further EB FM to the population of embryoid bodies after 24 hours of incubation in the EB FM.

Another aspect of the disclosure includes a method of generating at least one brain organoid, the method comprising
a. generating at least one brain organoid-sufficient embryoid body using any method described herein and/or generating at least one repaired brain organoid-sufficient embryoid body using any method described herein;
b. transferring the at least one brain organoid-sufficient embryoid body generated in step a) from the EB FM used in step a) to a neural induction medium without transferring any of the EB FM into the neural induction medium and incubating the at least one brain organoid-sufficient embryoid body in the neural induction medium for at least about 24 hours and up to about 96 hours, optionally 48 hours;
c. replacing the neural induction medium with expansion medium having a temperature of at least about 0° C. and up to about 12° C., optionally about 4° C. to about 6° C.; and
d. replacing the expansion medium with maturation medium after at least about 48 hours up to about 96 hours, optionally at least about 72 hours.

In some embodiments, the at least one brain organoid-sufficient embryoid body is incubated in the neural induction medium for about 48 hours. In some embodiments, the expansion medium is replaced with maturation medium after at least about 48 hours. In some embodiments, the expansion medium is replaced with maturation medium after about 72 hours. In some embodiments, the expansion medium is replaced with maturation medium after at least about 96 hours.

In some embodiments, the at least one brain organoid comprises astrocytes, neurons, oligodendrocytes, and/or microglia. In some embodiments, the at least one brain organoid comprises microglia cells and astrocytes. In some embodiments, the at least one brain organoid comprises microglia cells. In some embodiments, the at least one brain organoid comprises astrocytes.

In some embodiments, the method further comprises removing maturation medium and adding additional maturation medium as needed to ensure the nutrient medium does not become nutrient deprived and/or acidic. In some embodiments, the brain organoid formed in step d) can be maintained in maturation medium indefinitely, optionally for up to 270 days, or optionally for 90 days. In some embodiments, the brain organoid formed is maintained in maturation medium until spontaneous and synchronized neural network electrical activity is detectable in the brain organoid. In some embodiments, neural network electrical activity is detectable in the brain organoid at least 90 days after the generation of the at least one brain organoid-sufficient embryoid body.

In some embodiments, the neural induction medium comprises a cell culture medium, such as DMEM, and between about 0.1 μg/ml to 2 μg/ml heparin, optionally about 1 μg/ml. In some embodiments, the DMEM may be replaced with DMEM with F-12 Ham nutrient mixture (DMEM-F12), or DMEM-F12 with 4-(2-hydroxyethyl)-1-piperazineethanesulfonic acid (HEPES). When DMEM was used with these concentrations of heparin, EB yields were 75%, and this increased to 90% on average with DMEM-F12 with HEPES.

In some embodiments, the neural induction medium further comprises:
20 μg/ml recombinant human transferrin;
20 μg/ml recombinant human insulin;
20 ng/ml sodium selenite;
2 mM L-alanyl-L-glutamine dipeptide; and/or
0.2 mg/ml LAA.

In some embodiments, the expansion medium comprises an extracellular matrix, a cell culture medium, and lacks vitamin A.

In some embodiments, the expansion medium comprises
1:1 mixture of DMEM-F12 with HEPES:Neurobasal medium;
20 μg/μl recombinant human transferrin;
20 μg/μl recombinant human insulin;
20 ng/ml sodium selenite;
2 mM L-alanyl-L-glutamine dipeptide;
0.2 mg/ml LAA;
2× S15 without vitamin A supplement; and
2% v/v undiluted hESC-qualified Matrigel matrix.

In some embodiments, the expansion medium comprises:
DMEM-F12 with HEPES:Neurobasal medium (1:1 mixture), DMEM-F12, DMEM, BrainPhys, pure neurobasal, or pure neurobasal Plus;
B27 without vitamin A;
2-4% v/v undiluted extracellular matrix (Geltrex, Matrigel, etc.) or 10% v/v synthetic extracellular matric (VitroGel); and
L-alanyl-L-glutamine dipeptide or L-glutamine; and optionally LAA and/or N2.

In some embodiments, the maturation medium comprises an extracellular matrix, a cell culture medium, and vitamin A.

In some embodiments, the maturation medium comprises
1:1 mixture of DMEM-F12 with HEPES and Neurobasal medium;
20 ng/ml sodium selenite;
2 mM L-alanyl-L-glutamine dipeptide;
0.2 mg/ml LAA; and
2× S15 with vitamin A supplement.

In some embodiments, the maturation medium comprises:
DMEM-F12 with HEPES:Neurobasal medium (1:1 mixture), DMEM-F12, DMEM, BrainPhys, pure neurobasal, or pure neurobasal Plus;
B27 with vitamin A;
2-4% v/v undiluted extracellular matrix (Geltrex, Matrigel, etc.) or 10% v/v synthetic extracellular matrix (VitroGel); and
L-alanyl-L-glutamine dipeptide or L-glutamine; and optionally LAA and/or N2.

### Uses and Compositions for Use

Another aspect of the disclosure includes an embryoid body formation medium (EB FM) for generating at least one brain organoid-sufficient embryoid body, the EB FM comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin;
about 5 ng/ml to about 30 ng/ml of sodium selenite; and
about 1 ng/ml to about 200 ng/ml, optionally about 10 ng/ml to about 25 ng/ml, of thermostable fibroblast growth factor 2 (FGF2).

Another aspect of the disclosure includes use of an embryoid body formation medium (EB FM) for generating at least one brain organoid-sufficient embryoid body, the EB FM comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin;
about 5 ng/ml to about 30 ng/ml of sodium selenite; and
about 1 ng/ml to about 200 ng/ml, optionally about 10 ng/ml to about 25 ng/ml, of thermostable fibroblast growth factor 2 (FGF2).

In some embodiments, the EB FM further comprises:
about 1 μg/ml to about 1 mg/ml L-ascorbic acid (LAA);
about 5 mM to about 45 mM of sodium bicarbonate;
about 0.1 ng/ml to about 20 ng/ml of transforming growth factor beta-3 (TGF-β3) or about 1 ng/ml to about 200 ng/ml transforming growth factor beta-1 (TGF-β1); and/or
about 0.1 ng/ml to about 100 ng/ml neuregulin 1 (NRG1).

In some embodiments, the concentration of the glutamine in the EB FM is about 0.1 mM to about 10 mM. In some embodiments, the concentration of the glutamine in the EB FM is about 2 mM. In some embodiments, the concentration of the recombinant human insulin in the EB FM is about 20 μg/mL. In some embodiments, the concentration of the recombinant human transferrin in the EB FM is about 20 μg/ml. In some embodiments, the concentration of the sodium selenite in the EB FM is about 20 ng/ml. In some embodiments, the concentration of the LAA in the EB FM is about 0.2 mg/ml. In some embodiments, the concentration of the sodium bicarbonate in the EB FM is about 14.7 mM. In some embodiments, the concentration of the TGF-β3 in the EB FM is about 0.1 ng/ml. In some embodiments, the concentration of the NRG1 in the EB FM is about 0.1 ng/ml. In some embodiments, the concentration of the thermostable FGF2 in the EB FM is about 4 ng/ml. In some embodiments, the concentration of the thermostable FGF2 is about 50 ng/ml. In some embodiments, the concentration of the thermostable FGF2 in the EB FM is about 10 ng/ml to about 25 ng/ml.

The glutamine in the EB FM may be present in the EB FM in the form of any compound that is a source of glutamine, including for example L-alanyl-L-glutamine dipeptide.

In some embodiments, the cell culture medium minimally comprises DMEM. In some embodiments, the cell culture medium is or comprises DMEM, DMEM with F-12 Ham nutrient mixture (DMEM-F12), or DMEM-F12 with 4-(2-hydroxyethyl)-1-piperazineethanesulfonic acid (HEPES)

Another aspect of the disclosure includes use of an embryoid body formation medium (EB FM) for repairing at least one non-viable embryoid body, the EB FM comprising:
a culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin; and
about 5 ng/ml to about 30 ng/ml of sodium selenite.

Another aspect of the disclosure includes an embryoid body formation medium (EB FM) for repairing at least one non-viable embryoid body, the EB FM comprising:
a culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin; and
about 5 ng/ml to about 30 ng/ml of sodium selenite.

In some embodiments, the EB FM further comprises about 1 ng/ml to about 200 ng/ml of thermostable FGF2. In some embodiments, the EB FM comprises about 10 ng/ml to about 25 ng/ml of thermostable FGF2. In some embodiments, the EB FM comprises about 4 ng/ml to about 50 ng/ml of thermostable FGF2. In some embodiments, the concentration of the thermostable FGF2 in the EB FM is about 4 ng/ml. In some embodiments, the concentration of the thermostable FGF2 in the EB FM is about 50 ng/ml.

In some embodiments, the EB FM further comprises:
about 1 μg/ml to about 1 mg/ml L-ascorbic acid (LAA);
about 5 mM to about 45 mM of sodium bicarbonate;
about 0.1 ng/ml to about 20 ng/ml of transforming growth factor beta-3 (TGF-β3) or about 1 ng/ml to about 200 ng/ml transforming growth factor beta-1 (TGF-β1); and/or
about 0.1 ng/ml to about 100 ng/ml neuregulin 1 (NRG1).

In some embodiments, the concentration of the glutamine in the EB FM is about 0.1 mM to about 10 mM. In some embodiments, the concentration of the -glutamine in the EB FM is about 2 mM. In some embodiments, the concentration of the recombinant human insulin in the EB FM is about 20 μg/mL. In some embodiments, the concentration of the recombinant human transferrin in the EB FM is about 20 μg/ml. In some embodiments, the concentration of the sodium selenite in the EB FM is about 20 ng/ml. In some embodiments, the concentration of the LAA in the EB FM is about 0.2 mg/ml. In some embodiments, the concentration of the sodium bicarbonate in the EB FM is about 14.7 mM. In some embodiments, the concentration of the TGF-β3 in the EB FM is about 0.1 ng/ml. In some embodiments, the concentration of the NRG1 in the EB FM is about 0.1 ng/ml.

The glutamine in the EB FM may be present in the EB FM in the form of any compound that is a source of glutamine, including for example L-alanyl-L-glutamine dipeptide.

In some embodiments, the cell culture medium minimally comprises DMEM. In some embodiments, the cell culture medium is or comprises DMEM, DMEM with F-12 Ham nutrient mixture (DMEM-F12), or DMEM-F12 with 4-(2-hydroxyethyl)-1-piperazineethanesulfonic acid (HEPES).

Another aspect of the disclosure includes brain organoids produced by the processes described herein, brain organoid-sufficient embryoid bodies produced by the processes described herein, and/or brain organoid-sufficient embryoid bodies repaired by the processes described herein. In some embodiments, the brain organoids produced by the processes described herein comprise astrocytes, neurons, oligodendrocytes, and/or microglia. In some embodiments, the brain organoids produced by the processes described herein comprise microglia cells and astrocytes. In some embodiments, the brain organoids produced by the processes described herein comprise microglia cells. In some embodiments, the brain organoids produced by the processes described herein comprise astrocytes.

In some embodiments, neural network electrical activity is detectable in the brain organoid at least 90 days after the generation of the at least one brain organoid-sufficient embryoid body.

In some embodiments, the brain organoid is generated from iPSCs from a patient having Alzheimer's disease. Alzheimer's disease includes for example familial Alzheimer's disease, sporadic Alzheimer's disease, early-onset Alzheimer's disease and late-onset Alzheimer's disease. In some embodiments, the Alzheimer's disease comprises familial Alzheimer's disease, sporadic Alzheimer's disease, early-onset Alzheimer's disease, and/or late-onset Alzheimer's disease. In some embodiments, the Alzheimer's disease is sporadic Alzheimer's disease. In some embodiments, the brain organoid is generated from iPSCs from a patient suspected of having Alzheimer's disease, such as familial Alzheimer's disease, sporadic Alzheimer's disease, early-onset Alzheimer's disease and/or late-onset Alzheimer's disease. In some embodiments, the patient is suspected of having sporadic Alzheimer's disease.

Another aspect of the disclosure includes use of brain organoid generated using any of the methods described herein for diagnosing or predicting onset of Alzheimer's disease in a patient suspected of having or developing Alzheimer's disease.

Another aspect of the disclosure includes a method of diagnosing or predicting onset of Alzheimer's disease in a patient suspected of having or developing Alzheimer's disease, the method comprising:
a) collecting a sample from the patient,
b) isolating cells from the sample,
c) reprogramming the cells into induced pluripotent stem cells (iPSCs),
d) using the iPSCs to generate a brain organoid using any of the methods described herein, and
e) detecting the presence of markers associated with Alzheimer's disease in the brain organoid.

In an an embodiment, the markers associated with Alzheimer's disease comprise the presence of a phosphorylated tau protein in the brain organoid, as compared to a control, the presence of a phosphorylated tau protein at 24 kDa, an increase in the levels of ionized calcium-binding adapter molecule 1 (IBA1) as compared to a control, an increase in the levels of amyloid-beta peptides as compared to a control, and/or a reduction in pH as compared to a control.

Alzheimer's disease includes for example familial Alzheimer's disease, sporadic Alzheimer's disease, early-onset Alzheimer's disease and late-onset Alzheimer's disease. In some embodiments, the Alzheimer's disease comprises familial Alzheimer's disease, sporadic Alzheimer's disease, early-onset Alzheimer's disease and/or late-onset Alzheimer's disease. In some embodiments, the Alzheimer's disease is sporadic Alzheimer's disease.

In some embodiments, the control comprises a brain organoid generated using iPSCs which have not been generated from a patient having or suspected of having Alzheimer's disease (e.g., iPSCs generated from cells isolated from a patient that does not have and is not suspected of having Alzheimer's disease). In some embodiments, the control is a reference value (e.g., a value calculated based on historical values from patients known to not have Alzheimer's disease).

In some embodiments, the reduction in pH is detected by assessing whether there is a change in colouring of media comprising the brain organoid (e.g., yellowing of the media).

In some embodiments, the sample includes any biological sample from which cells can be isolated. In some embodiments, the cells are somatic cells. In some embodiments, the cells are progenitor cells. In some embodiments, the sample is a blood sample. In some embodiments, the cells are erythroid progenitor cells.

Methods for reprogramming progenitor cells are known in the art and reprogramming of cells can be performed using for example, commercially available reprogramming kits and/or methods such as those described in the Examples below.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

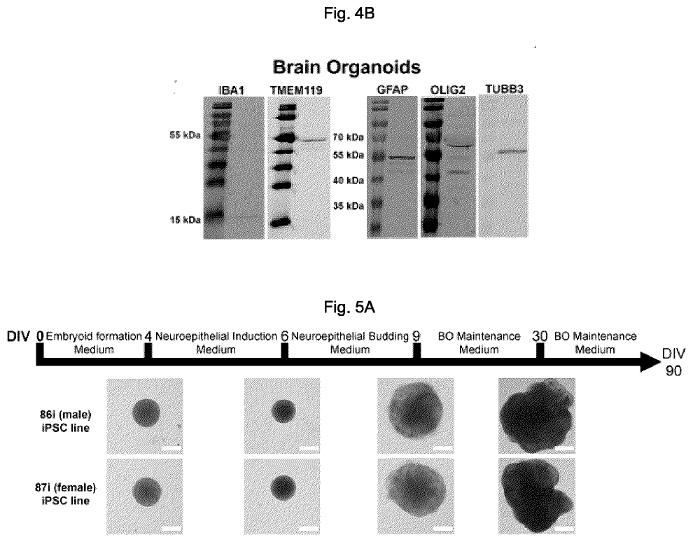

Microglia convert to a non-healthy (i.e., non-homeostatic) state when cultured in isolation in a dish (Gosselin et al., (2017)). This non-homeostatic state corresponds with abnormally high IBA1 and low TMEM119 levels (see FIG. 4) as well as the presence of endogenous signalling molecules, such as TNF (tumour necrosis factor)-alpha, that are not present in a healthy state (Lier et al., (2021)). Thus, isolated microglia cannot inform normal physiological responses to signalling molecules. Animal-derived microglia are equally limited in that they differ from human microglia in their expression of receptors, regulation of genes and the existence of signalling cascades (Yang et al., (2021); Gosselin et al., (2017)). To overcome the limitation of these models, brain organoids with putative healthy human microglia have been engineered herein.

Brain organoids are three-dimensional cultures of different cell types derived by manipulating differentiation of induced pluripotent stem cells (iPSCs). These organoids emulate the functions and cytoarchitectural changes associated with the developing human brain and, thus, offer a model to study the effects of exogenous and endogenous exposures on the developing human brain (from fetal to neonatal) without the confound of the parental placental environment. The initial stage of brain organoid growth involves the formation of an embryoid body (EB), which is a sphere of iPSCs. Experimental evidence suggest that inadequately formed EBs arise from substandard iPSCs. Using the same culture source of iPSCs, it is demonstrated herein that inadequate Ebs can be repaired within 24 hours post-exposure to a newly formulated in-house medium. Repaired Ebs can be differentiated into brain organoids using the protocol described herein. Importantly, these organoids are comprised of astrocytes, neurons, oligodendrocytes, and/or microglia. The latter cell type is often missing from brain organoid cultures. After 90 days in culture, brain organoids arising from repaired Ebs exhibit spatial distribution and expression levels of cell markers similar to brain organoids arising from undamaged Ebs. This suggests that repairing Ebs has no effect on the fate and function of cell and tissue architecture of brain organoids, thus mitigating any risk of ‘wasted’ cultures due to disruption of the EB, while achieving the cost efficiency needed to facilitate the wider adoption of this model by researchers.

### Materials and Methods

### Antibodies and Reagents

The following was purchased from Millipore Sigma (Oakville, ON, Canada): L-ascorbic acid (LAA), protease inhibitor cocktail (Cat #P8340), heparin and the Lowry assay kit (Peterson's modification). Erythroid Progenitor Reprogramming Kit, Gentle Cell Dissociation Reagent (GCDR), Human Pluripotent Stem Cell (hPSC) Genetic Analysis Kit, mTeSR™ Plus, STEMdiff™ Cerebral Organoid Kit and STEMdiff™ Trilineage Differentiation Kit, and Y-27632 (ROCK inhibitor) were purchased from STEMCELL Technologies (Vancouver, BC, Canada). Non-engineered basic fibroblast growth factor (FGF2), neuregulin 1 (NRG1) and trans-forming growth factor-β3 (TGF-β3) were purchased from Peprotech (Cranbury, NJ, USA). Recombinant human transferrin was purchased from InVitria (Fort Collins, CO, USA). Radioim-munoprecipitation assay buffer (RIPA) 10× was purchased from Cell Signaling Technologies (Whitby, ON, Canada). Antibodies and their suppliers are listed in Table 3. All other reagents were purchased from Fisher Scientific (Ottawa, ON, Canada).

TABLE 3
List of reagents and antibodies used.

| | | | |
|---|---|---|---|
| | Catalogue | Blotting | Immunohistochemistry |
| Target | Number | Dilution | Dilution |

Primary Antibodies

| | | | |
|---|---|---|---|
| Rabbit anti-TUBB3 | Millipore Sigma | 1:2,000 | 1:200 |
| | Cat#T2200 | | |
| mouse anti-GFAP | Millipore Sigma | 1:1,000 | 1:200 |
| | Cat#3893 | | |
| rabbit anti-OLIG2 | Millipore Sigma | 1:1,000 | 1:1000 |
| | Cat#AB9610 | | |
| Mouse anti-TMEM119 | BioLegend | 1:500 | 1:200 |
| | Cat#A16075D | | |
| Mouse anti-MBP | Millipore Sigma | N/A | 1:1000 |
| | Cat#AMAB91064 | | |
| Rabbit anti-SYN1 | Cell Signaling | 1:1000 | N/A |
| | Technology | | |
| | Cat#5297 | | |
| Mouse anti-PSD95 | ThermoFisher | 1:1000 | N/A |
| | Scientific | | |
| | Cat#MA1-045 | | |
| Rabbit anti-CX43 | Millipore Sigma | 1:1000 | N/A |
| | Cat#C6129 | | |

Secondary Antibodies

| | | | |
|---|---|---|---|
| IRDye ® 680RD anti-rabbit IgG | LI-COR | 1:20,000 | N/A |
| | Biosciences | | |
| | Cat#926-68071 | | |
| IRDye ® 680RD anti-mouse IgG | LI-COR | 1:20,000 | N/A |
| | Biosciences | | |
| | Cat#926-68070 | | |
| IRDye ® 800CW anti-rabbit IgG | LI-COR | 1:20,000 | N/A |
| | Biosciences | | |
| | Cat#926-32211 | | |
| IRDye ® 800CW anti-mouse | LI-COR | 1:20,000 | N/A |
| IgG | Biosciences | | |
| | Cat#926-32210 | | |

| | |
|---|---|
| Reagent | Catalogue Number |

Growth Factors

| | |
|---|---|
| Non-engineered FGF2 | PeproTech Cat#100-18B |
| Thermostable (engineered) | ThermoFisher Scientific Cat#PHG0369 |
| FGF2 | |
| NRG1 | PeproTech Cat#100-03 |
| TGF-β3 | PeproTech Cat#100-36E |

Abbreviations: BIII-Tubulin (TUBB3), basic fibroblast growth factor (FGF2), glial fibrillary acidic protein (GFAP), immunoglobulin G (IgG), myelin basic protein (MBP), neuregulin 1 (NRG1), oligodendrocyte transcription factor 2 (OLIG2), transforming growth factor (TGF), transmembrane protein 119 (TMEM119).

Induced Pluripotent Stem Cell (iPSC) Maintenance

Three human iPSC lines were used for all experiments. Karyotypically normal UCSD086i-6-3 and UCSD087i-6-4 iPSC lines were purchased from WiCell (Madison, WI, USA). Karyotypically normal USASK-29M-C-0-1 iPSC line was reprogrammed from erythroid progenitor cells at the University of Saskatchewan following instructions in a commercial kit (STEMCELL Technologies) with a minor modification during transfection. The transfection instructions in the Epi5™ Episomal iPSC Reprogramming Kit for human CD34+ cells were followed, rather than using the electroporator and settings described in the kit from STEMCELL Technologies. Karyotype of iPSCs were monitored using the hPSC Genetic Analysis Kit and pluripotency of iPSCs was confirmed using the STEMdiff™ Trilineage Differentiation Kit. iPSCs were cultured in feeder-free conditions on 6-well tissue culture plates coated with Matrigel™ hESC-qualified matrix (Corning™ 354277) or Geltrex™ hESC-qualified matrix and maintained with mTeSR™ Plus medium following manufacturer's instructions or EB FM 5 following the same instructions at 37° C. in humidified 5% CO2 and 95% air atmosphere.

### Generation of Cerebral Organoids

Human three-dimensional brain organoids were generated as described elsewhere (Lancaster and Knoblich, 2014) with modifications, or using commercial kits from STEMCELL Technologies. The modifications to the Lancaster and Knoblich, 2014 protocol included modifications to the components of the EB FM (see Table 4), neural induction medium, expansion medium and maturation medium used, and to the expansion steps of the protocol used in Lancaster and Knoblich, 2014. The neural induction medium, expansion medium and maturation medium used herein lack minimal essential medium-non-essential amino acids (MEM-NEAA) and replace N2 with insulin, transferrin, selenite, and L-ascorbic acid. Further, in the method used herein, the extracellular matrix is diluted in expansion medium rather than encapsulating the organoid itself in extracellular matrix, which is a technical barrier. Additionally, in the expansion and maturation medium used herein, B27 is replaced with S15 without vitamin A, and these media lack antibiotics.

Briefly, iPSCs were incubated with 0.5 mM ethylenediaminetetraacetic acid (EDTA) for four minutes and lifted off the plate with a cell scraper. iPSCs were centrifuged, pelleted, and resuspended at 9×10⁴ iPSCs/ml in one of the EB formation media (FM) described in Table 4 with 10 UM ROCK inhibitor.

TABLE 4
Formulations of EB FM used during day 0-4 of brain organoid generation.

| | EB FM 2 | | | |
|---|---|---|---|---|
| EB FM 1 | (Lancaster) | EB FM 3 | EB FM 4 | EB FM 5 |
| EB Formation | DMEM-F12 with | DMEM-F12 | DMEM-F12 | DMEM-F12 |
| Medium from | HEPES | with HEPES | with HEPES | with HEPES |
| STEMdiff ™ | (Gibco ™ 11330- | (Gibco ™ 11330- | (Gibco ™ 11330- | (Gibco ™ 11330- |
| Cerebral | 032) | 032) | 032) | 032) |
| Organoid Kit | 1X GlutaMAX ™ | 2 mM L-alanyl- | 2 mM L-alanyl- | 2 mM L-alanyl- |
| | | L-glutamine | L-glutamine | L-glutamine |
| | | dipeptide ^{d} | dipeptide ^{d} | dipeptide ^{d} |
| | 3% v/v ESC- | 20 μg/ml | 20 μg/ml | 20 μg/ml |
| | qualified FBS | recombinant | recombinant | recombinant |
| | | human insulin ^{c} | human insulin ^{c} | human insulin ^{c} |
| | | 20 μg/ml | 20 μg/ml | 20 μg/ml |
| | | recombinant | recombinant | recombinant |
| | | human | human | human |
| | | transferrin ^{c} | transferrin ^{c} | transferrin ^{c} |
| | | 20 ng/ml sodium | 20 ng/ml sodium | 20 ng/ml sodium |
| | | selenite ^{c} | selenite ^{c} | selenite ^{c} |
| | 20% v/v KOSR | 0.2 mg/ml LAA ^{c} | 0.2 mg/ml LAA ^{c} | 0.2 mg/ml LAA ^{c} |
| | 1X MEM-NEAA | 14.7 mM | 14.7 mM | 14.7 mM |
| | | Sodium | Sodium | Sodium |
| | | bicarbonate ^{a} | bicarbonate ^{a} | bicarbonate ^{a} |
| | 0.0007% v/v 2- | 0.1 ng/ml TGF- | 0.1 ng/ml TGF- | 0.1 ng/ml TGF- |
| | mercaptoethanol | β3 ^{b} | β3 ^{b} | β3 ^{b} |
| | | 0.1 ng/ml NRG1 ^{a} | 0.1 ng/ml NRG1 ^{a} | 0.1 ng/ml NRG1 ^{a} |
| | 4 ng/ml non- | | 4 ng/ml | 50 ng/ml |
| | engineered FGF2 | | thermostable | thermostable |
| | | | FGF2 | FGF2 |

Abbreviations:
4-(2-hydroxyethyl)-1-piperazineethanesulfonic acid (HEPES),
basic fibroblast growth factor (FGF2),
Dulbecco's modified Eagle medium with F-12 Ham nutrient mixture (DMEM-F12),
embryoid body (EB),
embryonic stem cell (ESC),
fetal bovine serum (FBS),
formation media (FM),
KnockOut ™ Replacement Serum (KOSR),
minimal essential medium- non-essential amino acids (MEM-NEAA),
neuregulin 1 (NRG1),
transforming growth factor (TGF)
Note:
final concentration of sodium bicarbonate is 29 mM (Gibco ™ medium includes sodium bicarbonate).
^{a} In subsequent experiments, it was observed that excluding this reagent does not alter EB yield, nor change the cellular composition of brain organoids
^{b} In subsequent experiments, it was observed that 0.1 ng/ml TGF-β3 can be replaced by TGF-β1 at concentrations 0.1 ng/ml to 100 ng/ml.
TGF-β3 can also be used at these higher concentrations.
^{c} In subsequent experiments, it was observed that a range of concentrations for these reagents create viable Ebs, although they cannot be absent from the EB FM. These concentrations were chosen as a trade-off between cost and growth rate of Ebs.
^{d} L-alanyl-L-glutamine dipeptide can be replaced by L-glutamine. Although, it should be noted that the stability of L-glutamine is affected by light exposure, pH, and temperature.

iPSCs resuspended in 100 μl of EB FM were seeded in 96-well ultra-low attachment round-bottom plates (day 0). 24 h later (day 1), Ebs were observed in wells containing EB FM 1,3-5, and an additional 100 μl of the same EB FM, or in certain experiments EB FM 3 or 6, were added to each well. At 72 h incubation (day 3), 100 μl of the EB FM used on day 1 was added to each well. On day 4, using 200 μl wide-bore pipette tips, one EB was transferred to each well of a 24-well ultra-low attachment plate by pipetting up 30 μl of media with the EB, letting the EB fall to the opening of the pipette tip, and submerging the tip in 1 ml neural induction media (DMEM-F12 with HEPES, 20 μg/ml recombinant human transferrin, 20 μg/ml recombinant human insulin, 20 ng/ml sodium selenite, 2 mM L-alanyl-L-glutamine dipeptide, 0.2 mg/ml LAA, 1 μg/ml heparin). This process prevents the transfer of EB FM (which prevents neuroectoderm formation) to the neural induction media, as well as fusion of brain organoids. On day 6, media was replaced with 500 μl of ice-cold expansion media (1:1 mixture of DMEM-F12 with HEPES:Neurobasal medium, 20 μg/μl recombinant human transferrin, 20 μg/μl recombinant human insulin, 20 ng/ml sodium selenite, 2 mM L-alanyl-L-glutamine dipeptide, 0.2 mg/ml LAA, 2×S15 without vitamin A supplement and 2% v/v undiluted hESC-qualified Matrigel matrix). On day 9, expansion media was replaced with 1 ml of maturation media (1:1 mixture of DMEM-F12 with HEPES and Neurobasal medium, 20 ng/ml sodium selenite, 2 mM L-alanyl-L-glutamine dipeptide, 0.2 mg/ml LAA, 2×S15 with vitamin A supplement) and incubated on a plate shaker set at 0.118 g. Once a week, media was replaced with 750 μl of maturation media, followed by an additional 500 μl being added four days later. Brain organoids were used for immunoblotting and immunohistochemistry on day 90. Brightfield images of brain organoids were taken with an Olympus CK53 microscope.

TABLE 5
Formulations of S15 used during day 7-9, and beyond day 9, of brain organoid generation.

| | | Final concentration | | Stock (mg/ml), | |
|---|---|---|---|---|---|
| | | in media (2X) | | prior to | |
| Reagent | Cat# | μg/ml | μM | conditions | Stock storage |
| Corticosterone | Millipore Sigma | 0.04 | 0.116 | 2 | −80° C. in 100% |
| | Cat#C2505 | | | | ethanol |
| Linoleic acid | Millipore Sigma | 2 | 7 | 100 | |
| | Cat#L1012 | | | | |
| Linolenic acid | Millipore Sigma | 2 | 7 | 100 | |
| | Cat#L2376 | | | | |
| Lipoic acid | Millipore Sigma | 0.094 | 0.4 | 4.7 | |
| (thioctic acid) | Cat#L2376 | | | | |
| Progesterone | Millipore Sigma | 0.0126 | 0.04 | 3.2 | |
| | Cat#P8783 | | | | |
| D,L-α-Tocopherol | Millipore Sigma | 2 | 4.6 | 100.0 | |
| (Vitamin E) | Cat#T3251 | | | | |
| D,L-α-Tocopherol | Millipore Sigma | 2 | 4.2 | 100.0 | |
| acetate | Cat#T3001 | | | | |
| Retinol acetate | Millipore Sigma | 0.2 | 0.4 | 20.0 | |
| (Vitamin A) ^{a} | Cat#R7882 | | | | |
| Retinol, all trans | Millipore Sigma | 0.2 | 0.6 | 10.0 | |
| (vitamin A) ^{a} | Cat#R1512 | | | | |

Other

| | | | | | |
|---|---|---|---|---|---|
| T3 (triiodo-L- | Cayman | 0.004 | 0.0052 | 2.0 | −20° C., light |
| thyronine) | Chemicals | | | | sensitive |
| | Cat#16028 | | | | |
| Ethanolamine | Millipore Sigma | 2 | 32 | 1000 | Liquid, room |
| | Cat#E9508 | | | | temperature |
| D(+)-Galactose | Millipore Sigma | 30 | 166 | | Solid, room |
| | Cat#G0625 | | | | temperature |
| L-Carnitine | Millipore Sigma | 0 | 24 | | Solid, room |
| | Cat#C0283 | | | | temperature |
| Glutathione | Millipore Sigma | 2 | 6.4 | | Solid, 2-8° C. |
| (Reduced) | Cat#G6013 | | | | |
| Putrescine | Millipore Sigma | 32.2 | 366 | | Solid, room |
| | Cat#P5780 | | | | temperature |

^{a} Exclude from formulation to make S15 without Vitamin A

### Cryosectioning, Immunoblotting and Immunostaining

iPSC-derived microglia and organoids were collected and homogenized in RIPA buffer containing protease inhibitor cocktail. Samples were triturated with a one ml pipette, homogenized using a 22-gauge needle, and sonicated. Protein concentration was quantified using the Lowry (Folin-Ciocalteu phenol reagent) assay, equalized to 0.5 μg/μl in 1% loading buffer (0.2 M Tris pH 6.8, 40% glycerol, 8% sodium dodecyl sulfate, 20% β-mercaptoethanol, 0.4% bromo-phenol blue), and heated at 100° C. for 10 min. Protein (15 μg or 20 μg per lane) was loaded on 12% polyacrylamide gels and resolved proteins were electroblotted onto a nitrocellulose membrane. After blocking in 5% w/v skim milk powder in TRIS-buffered saline (TBS: 250 mM Tris pH 7.4, 1.37 M NaCl) for one h, membranes were incubated overnight at 4° C. with primary antibodies (Table 3) diluted in 5% w/v skim milk powder in TBS-T (TBS with 0.1% Tween®20). After three washes with TBS-T over 30 min, secondary fluorescently labelled antibodies (Table 3) were added for one h, followed by three washes. Proteins were visualized with a LI-COR Odyssey® Imager and manufacturer's software (Image Studio 5.3.5, LI-COR Biosciences, Lincoln, NE, USA).

Brain organoids were transferred to a conical tube, washed thrice with sterile PBS, stored in 10% neutral buffered formalin for 16 h at 4° C., and then washed another three times with 0.1% Tween®20 in PBS (PBS-T). 24 h later, fixed organoids were transferred to 30% sucrose in PBS for approximately 16 h until they settled onto the bottom of a conical tube. Organoids were transferred to a mould and embedded in optimal cutting temperature (OCT) compound. Tissues were quickly frozen and cut into 20 μm sections using a Leica CM1950 cryostat and microtome and mounted on SuperFrost™ Plus microscope slides. Organoid sections were outlined with a Pap pen, washed thrice with PBS-T at 25° C. for 15 min to remove OCT, immersed in blocking solution (5% normal donkey serum in PBS-T) for one h, and incubated for 16 h at room temperature with primary antibodies (Table 3) diluted in 5% BSA in PBS-T. After six washes with PBS-T over one h, organoid sections were incubated with secondary antibodies (Table 3) at room temperature in a humidified chamber for two h, followed by three additional washes. Slides were air-dried, ProLong™ Glass Antifade Mountant with Nu-cBlue™ added, and a coverslip placed on top. Slides were then cured for 24 h at room temperature prior to imaging on a Zeiss Axiolmager M.1 widefield or Olympus FV1000 confocal microscope. iPSC-derived microglia were stained on chamber slides in a manner similar to brain organoids.

### Data Analysis

Data (means±standard deviation (SD)) were analyzed using the Kruskal-Wallis test, followed by the Tukey's post-hoc test. Significance was established at P<0.05. Independent experiments are defined as assays performed on different days.

### Results

### Thermostable FGF2 Increases EB Yield

Several culture media for the generation of Ebs were compared. These media included a commercial kit (EB FM 1), the most widely used medium, e.g., the formulation described in Lancaster & Knoblich (2014) (EB FM 2), and newly formulated in-house media with different concentrations of thermostable FGF2 (EB FM 3-5). EB FM 5 yielded significantly more (e.g., 96%) viable Ebs than any of the other formulations tested (e.g., 10-25%) and 0% Ebs formed when using the widely used medium EB FM 2 (Table 6). Experiments consisted of 8-16 replicates, with replicates spanning different days to account for both day- and plate-bias. Three different progenitor iPSC lines were also tested.

Representative images of data in Table 6 are shown in FIG. 1A-B. EB FM 1 formed multiple inadequate Ebs, while EB FM 3-5 more often formed the expected single, round EB (FIG. 1A). In all attempts, EB FM 2 did not form stable Ebs, as they lost structural integrity within 48 h (FIG. 1B).

TABLE 6
Number of Ebs produced when grown in different EB FMs

| | % Viable Ebs at 96 h | |
|---|---|---|
| EB FM | (viable Ebs/total Ebs | |
| EB FM 1 (Commercial kit) | 10.1% | (99/984) |
| EB FM 2 (Most cited) | 0% | (0/192) |
| EB FM 3 | 11.5% | (12/104) |
| EB FM 4 | 25% | (32/128 |
| EB FM 5 | 96.8% | (422/436) |

### Unsuitable Ebs can be Repaired by EB FM 3 and 5

Poor EB yields have been attributed to low cell seeding densities or poor quality iPSCs (Lancaster & Knoblich, 2014). To demonstrate how key nutrients are critical to EB formation, it was tested whether the new media formulations described herein could repair unsuitable Ebs using the same pool of iPSCs (Table 7). EB FM 3 and 5 were able to repair unsuitable Ebs generated with EB FM 1. Furthermore, components of EB FM 5 were only required for the first 24 h of EB formation. Representative images of brain organoid repair are shown in FIG. 1C. While the same number of independent experiments were conducted across all conditions, more Ebs were seeded in conditions where lower EB yields were expected.

TABLE 7
Specific EB FM formulations can repair inadequately formed Ebs

| | | % viable Ebs at 24 h | | % viable Ebs at 96 h | |
|---|---|---|---|---|---|
| EB FM (0-24 h) | EB FM (24-96 h) | (viable Ebs/total Ebs) | | (viable Ebs/total Ebs) | |
| EB FM 1 | EB FM 1 | 6.7% | (13/192) | 6.7% | (13/192) |
| (Commercial kit) | (Commercial kit) | | | | |
| EB FM 1 | EB FM 3 | 2.6% | (7/264) | 76.9% | (203/264) |
| (Commercial kit) | | | | | |
| EB FM 3 | | 10.5% | (16/152) | 10.5% | (16/152) |
| EB FM 4 | | 40% | (97/240) | 40% | (96/240) |
| EB FM 5 | | 89.0% | (114/128) | 89.0% | (114/128) |
| EB FM 1 | EB FM 5 | 1.1% | (3/284) | 78.5% | (223/284) |
| (Commercial kit) | | | | | |

### Repaired Ebs Exhibit Levels of Cell Markers Similar to Non-Repaired Ebs Seeded in the Same Culture Media

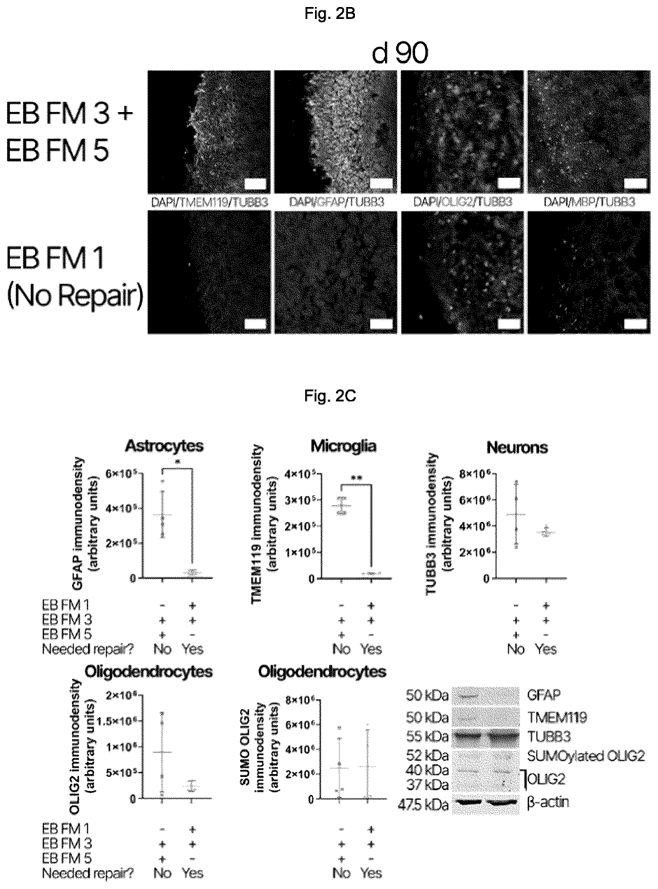

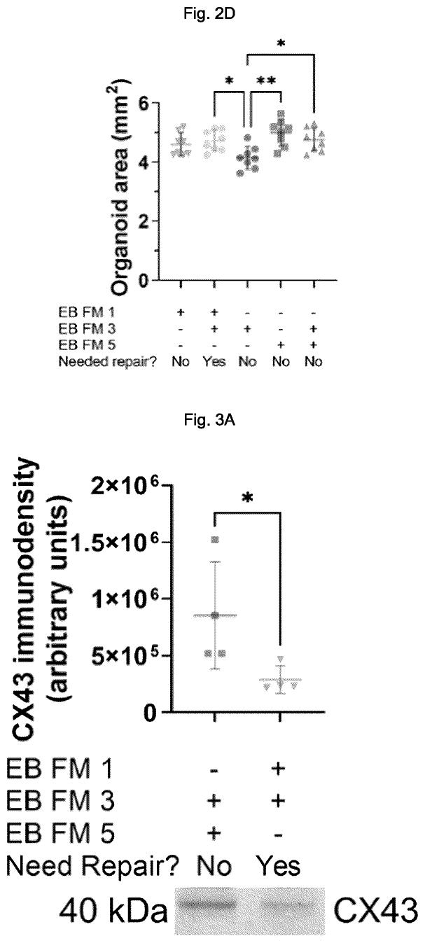

To determine whether repairing Ebs alters subsequent organoid formation, Ebs that were repaired and Ebs that did not need repair were differentiated into cerebral organoids using a self-patterning protocol. Both repaired Ebs and Ebs not needing repair displayed all visual milestones of brain organoid development (FIG. 2A).

The cellular composition of a subset of organoids was assessed. Glial markers, ‘trans-membrane protein 119’ (TMEM119), ‘glial fibrillary acidic protein’ (GFAP), oligodendrocyte transcription factor 2′ (OLIG2) and ‘myelin basic protein’ (MBP) were stained in brain organoids seeded in EB FM 5 and maintained in EB FM 3 (FIG. 2B, top). TMEM119 and GFAP were not present in brain organoids seeded in EB FM 1 alone, i.e., they did not need repair (FIG. 2B, bottom). To confirm whether the detection of glial markers was due to non-specific binding, immunodensity of TMEM119, GFAP and OLIG2 bands were measured in protein lysates (FIG. 2C). The immunodensity of GFAP and TMEM119 were higher in brain organoids seeded in EB FM 5 and maintained in EB FM 3 compared to repaired brain organoids seeded in EB FM 1. No difference in the immunodensity of TUBB3 and OLIG2 were detected in either group. Representative immunoblots are shown. Brain organoids grown exclusively in EB FM 4 were smaller than organoids grown in the other combinations of EB FM tested (FIG. 2D).

Brain Organoids with Astrocytes and Microglia have Higher Levels of Astrocytic Synaptic Protein CX43 and Presynaptic Protein SYN1

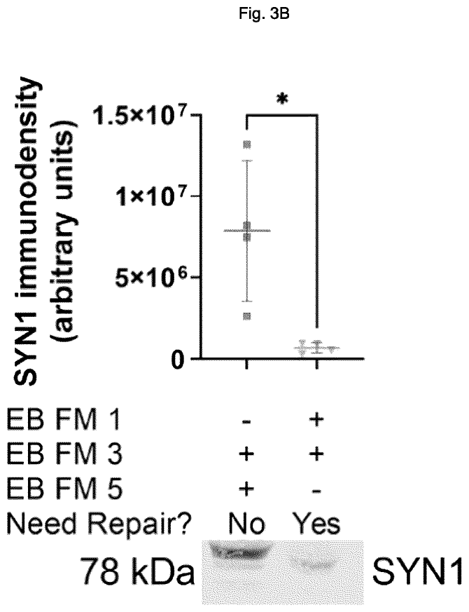

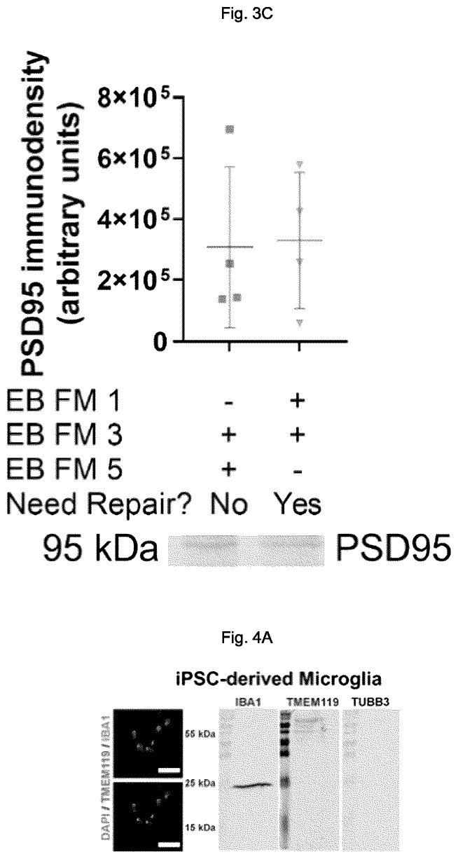

As astrocytes and microglia are involved in synaptic maintenance, the same protein lysate was used to measure the immunodensity of astrocytic synaptic protein ‘connexion 43’ (CX43), as well as presynaptic protein ‘synapsin 1’ (SYN1) and ‘postsynaptic density protein 95’ (PSD95). The immunodensity of CX43 and SYN1 was higher in brain organoid cultures with astrocytes and microglia compared to cultures without these cells (FIG. 2A, B). There was no difference in the immunodensity of PSD95 (FIG. 3C). Representative immunoblots are shown.

### DISCUSSION

Guidelines in brain organoid protocols suggest that EB yield is dependent on the quality of iPSC cultures as well as iPSC seeding density during EB formation (Lancaster & Knoblich, 2014). Using commercial kits (rated for a 60-80% yield), suitable Ebs could only be generated in ˜10% of attempts and no Ebs could be generated when following the protocol described in Lancaster & Knoblich (2014). It is not clear why Ebs yields were different between these two methods, as the commercial kit is based on the media formulations in the Lancaster & Knoblich (2014) report. However, it is noted that the commercial kit is serum-free, unlike the formulations in Lancaster & Knoblich (2014), and this serum-free condition was more similar to the culture media the iPSCs are routinely grown in, suggesting that media formulations could be a primary factor for suitable EB formation.

The series of experiments using the same pool of iPSCs explain why Ebs may fail to form. After confirming the pluripotency of the karyotypically-normal iPSCs, a variety of different seeding densities were tested. These attempts did not increase EB yield, demonstrating that seeding density is not a determining factor of EB formation. Next, the effects of different formulations of EB FM on EB formation were investigated and now it was confirmed that EB FM is the key determiner of suitable EB formation. Importantly, it was observed that certain formulations of EB FM can repair unsuitable Ebs, and subsequently restore the normal development of brain organoids. Thus, the EB FM formulation is the primary factor of suitable EB formation, not iPSC quality or their seeding density.

Brain organoids derived from certain EB FM formulations expressed consistently detectable levels of glial markers TMEM119, GFAP and OLIG2 (FIG. 4A-B). This is in clear contrast with brain organoids derived from the EB FM in the commercial kit. This observation of a lack of glial markers in cultures based on commercial kits is keeping inline with other studies, as mRNA expression of astrocytic and oligodendroglial-specific markers is commonly absent at 90 days in cultures and only begins to dramatically increase around day 180 (Sivitilli et al., 2020); this suggests that repairing Ebs has no effect on either the fate and function of cells in brain organoids or on their cytoarchitecture. Furthermore, the absence of microglial markers, including TMEM119 and IBA1, is common in self-patterned brain organoid protocols (Sivitilli et al., 2020; Tanaka et al., 2020), although de Witte and their colleagues reported that modification to the Lancaster & Knoblich (2014) protocol between days 6-12, namely using 0.1 μg/mL instead of 1 μg/mL heparin in the neural induction media, replacing L-glutamine and with GlutaMAX and keeping the brain organoids in the neural induction media for an extra 3 days, results in the presence of IBA1+ cells (e.g., microglia) in brain organoids and these organoids express TMEM119 mRNA (Gumbs et al., 2022; Ormel et al., 2018). Interestingly, microglia-containing brain organoids express astrocytic proteins, such as GFAP, and oligodendroglial mRNA, such as OLIG2, as early as day 52 (Ormel et al., 2018), supporting a hypothesis that microglia are critical to the maturation of other brain cell types (Cowan and Petri, 2018; Lukens and Eyo, 2022). It is noted that the only difference between the brain organoid groups in the present data was the media used prior to day 6, highlighting a pertinent knowledge gap about the factors and their respective contributions to the presence and maturation of glial cells in brain organoid cultures.

The data described herein indicates astrocytes and microglia in brain organoids display their normal functions in synaptic maintenance. As expected, the data shows that higher levels of astrocytic synaptic protein CX43 is associated with GFAP immunodensity (Droguerre et al., 2019). It has been reported that the presence of microglia bolsters the expression of presynaptic protein SYN, which is inline with the present data (Cristovão et al., 2014). Interestingly, no differences in the immunodensity of PSD95 were observed, which may be explained by PSD95 having a brain-region specific expression profile (Curran et al., 2021). The low expression of PSD95 in non-brain region-specific organoids are inline with whole human brain tissue lysate (Abcam, n.d.). This data certainly warrants investigating the contributions of microglia and astrocytes to synaptic formation, which up until the generation of brain organoids such as those described herein, has been difficult due to the off-target effects of ablating these cells in animal models. Importantly, it is shown herein that the brain organoids described herein can be used to study whether treatments or genetic backgrounds modulate the levels of synaptic proteins.

This study is the first to demonstrate EB FM is a factor more critical to EB yield than iPSC seeding density and iPSC quality. In fact, the culture medium formulation EB FM 5 described herein reproducibly yields 95%+ viable Ebs, which is far in excess of other media formulations, including the most commonly used commercial formulation. It is also demonstrated for the first time herein that Ebs can be repaired, and that this restoration has no effect on the generation of brain organoids. Thus, it is possible to avoid discarding what has until now been viewed as ‘wasted’ cultures due to disruption of the EB. Not only does this rescue cost-, time-, and labour-intensive work for those laboratories already invested in organoid research, it increases the potential for others to adopt organoid platforms to take their current research into pre-translational contexts that may have otherwise been considered to be too risky and cost-prohibitive. The evidence suggests that the incorporation of microglia into brain organoids allows for these cultures to transition into a state with a more proteostatic signature, giving researchers a model more representative of human brain physiology.

### Example 2

Brain organoids (BOs) are three-dimensional cultures of neurons and glia generated from induced pluripotent stem cells (iPSCs) that spontaneously distribute and layer themselves in a manner similar to the human brain. After enough time in culture, BOs also exhibit neural network activity, indicating they are brain-like. Several BO protocols exist, although organoids generated with these protocols do not often have microglia (the innate immune cells of the brain), limiting the translational potential of data gathered from such models. BOs are often described as models of the human brain and highly variable cultures, but the tissues have not been benchmarked to primary tissues. The brain organoid protocol described herein gives rise to homeostatic microglia innately. Herein, the developmental timeline of the BOs described herein was mapped, and in particular, whether there were any notable protein variations between cultures was tested, and the protein profile of BOs were benchmarked against human and mouse brain tissue.

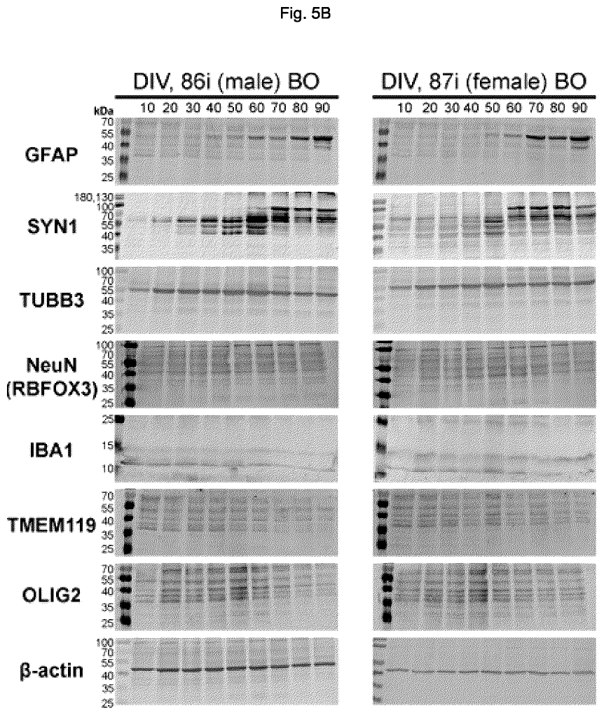

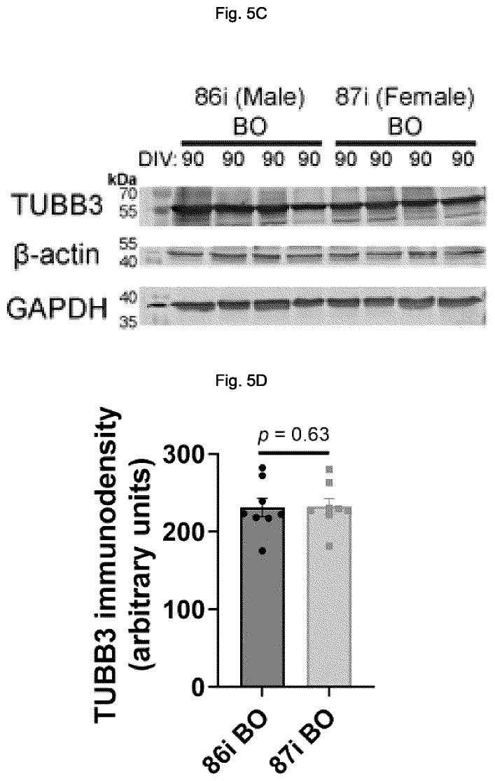

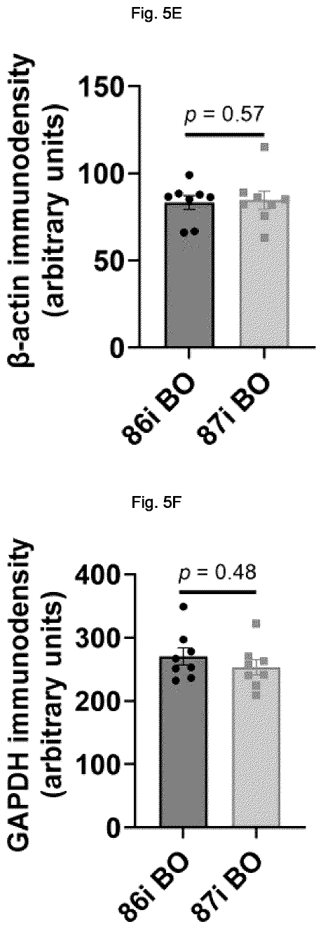

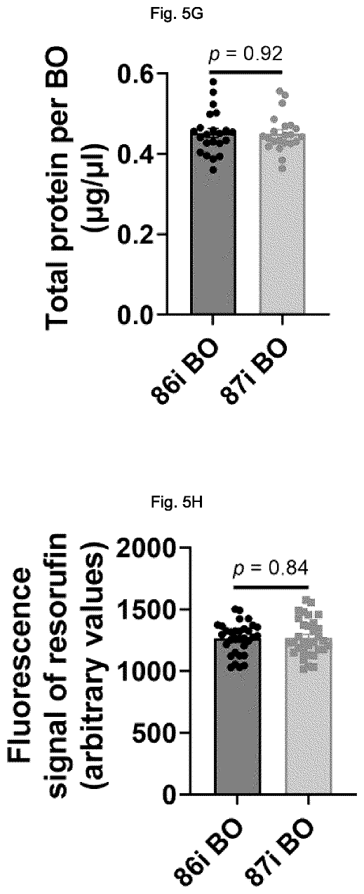

BOs generated by a tightly-regulated standard operating protocol exhibit negligible variability. FIGS. 5A-H demonstrate the developmental time course of the present disclosure's unique brain organoid platform. The methodology described herein allows for cost- and time-efficient analysis of brain organoids at many acute timepoints. Other protocols are not readily permissive to such analysis. Not only is the presence of microglial proteins demonstrated in FIGS. 5A-H, these figures also establish that synapses (SYN1) and astrocytes (GFAP) began to form between day 40-60. FIGS. 5A-H also show that these same proteins matured at day 70 (SYN1) and day 90 (GFAP), as represented by their appearance at the characteristic molecular weight (˜80 kDa) and the presence of isoforms (multiple bands), respectively. Similarly, oligodendrocytes (OLIG2) had a maximal expression between day 40-50. Usually, oligodendrocytes, astrocytes and synapses are described to be present after 6 months in culture. Thus, the presently described media formulation seems to allow organoids to develop at twice the speed, which is highly desirable. It is also demonstrated that the presently described media formulations and corresponding methodology resulted in highly similar brain organoids, as demonstrated by the data in FIGS. 5C-G showing low variability. This low variability was further corroborated by FIG. 5B showing the protein banding patterns of organoids were similar, and by FIG. 5A showing the organoids developed similar visual hallmarks-regardless of cell line used. This supports the optimized media formulations being key to the consistent development of organoids, as it is often cited that brain organoids are dissimilar between cell lines.

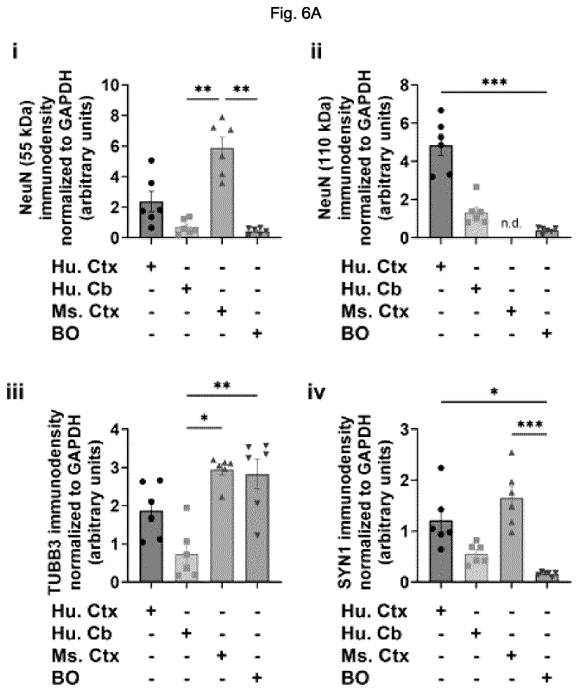

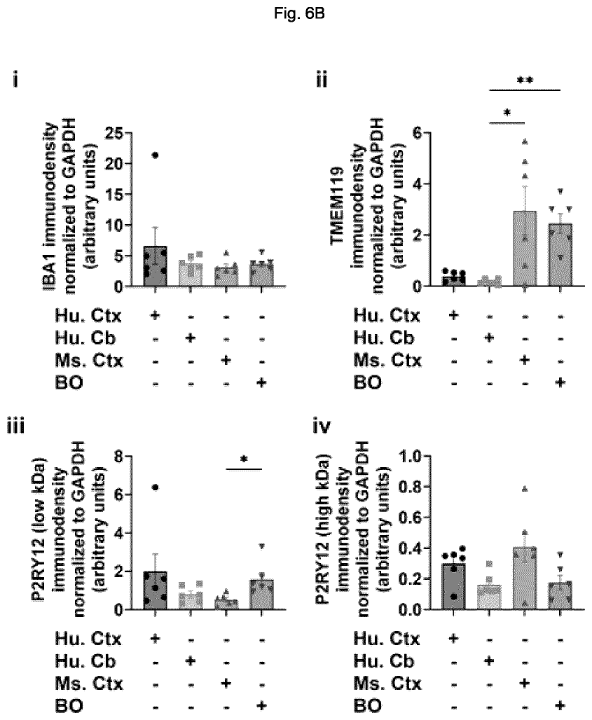

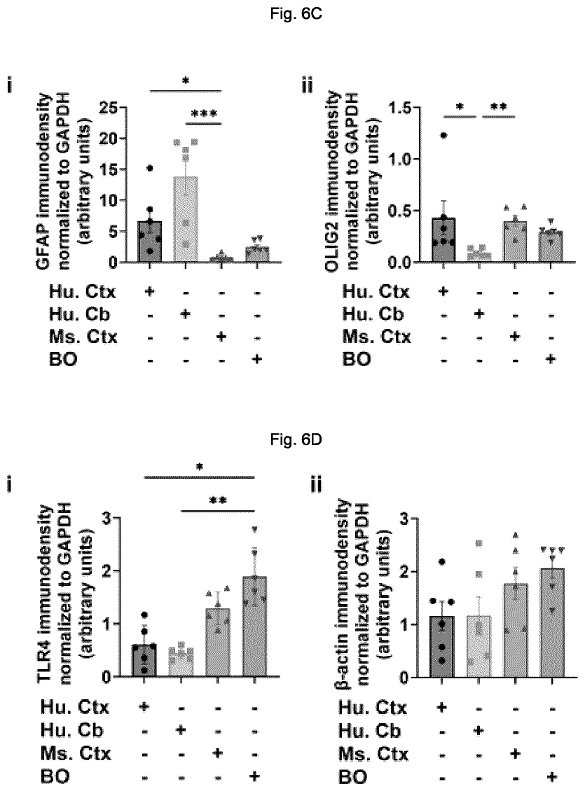

BOs often express proteins at molecular weights and levels similar to the adult human brain. FIGS. 6A-D show that, with a few exceptions, the presently described brain organoids generated using the presently described methods tend to be more similar to human brain tissue than mouse brain tissue. Again, as many of these protein markers are not present in other protocols at this time point (e.g., GFAP, IBA1), it supports the presently described methodology as a desirable model of the adult brain.

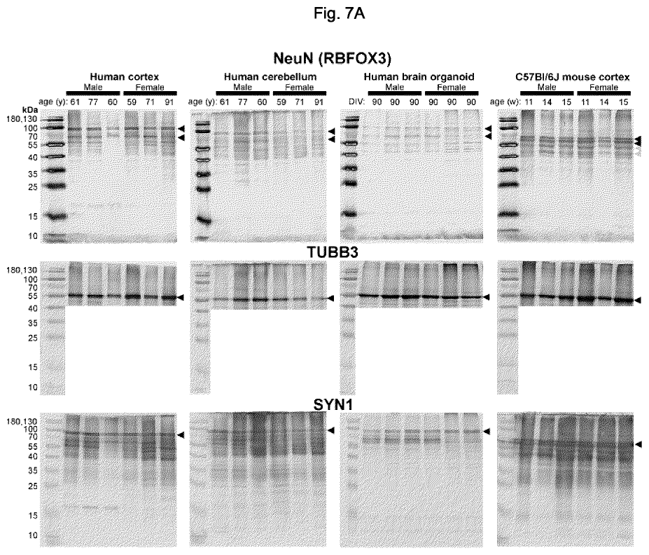

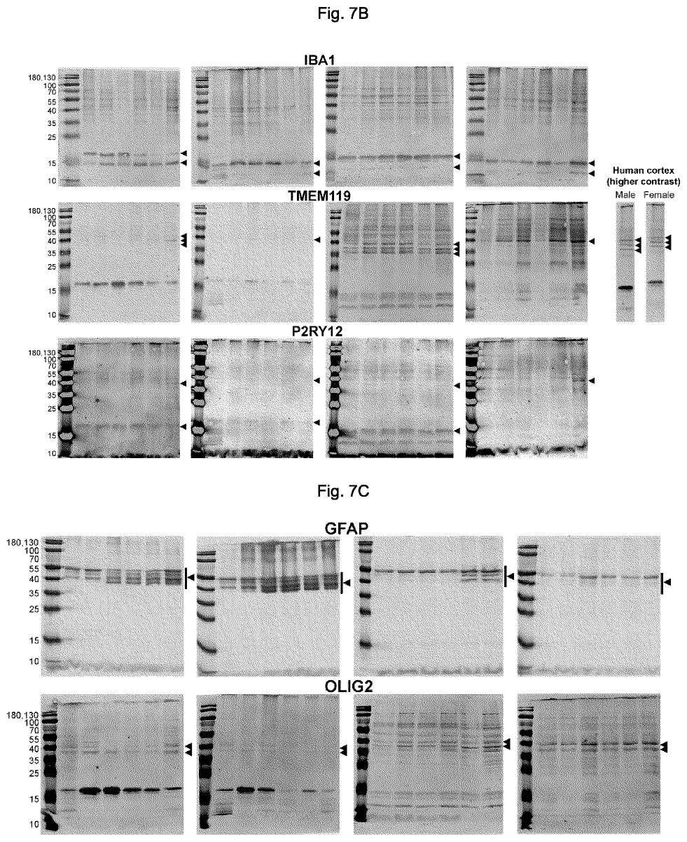

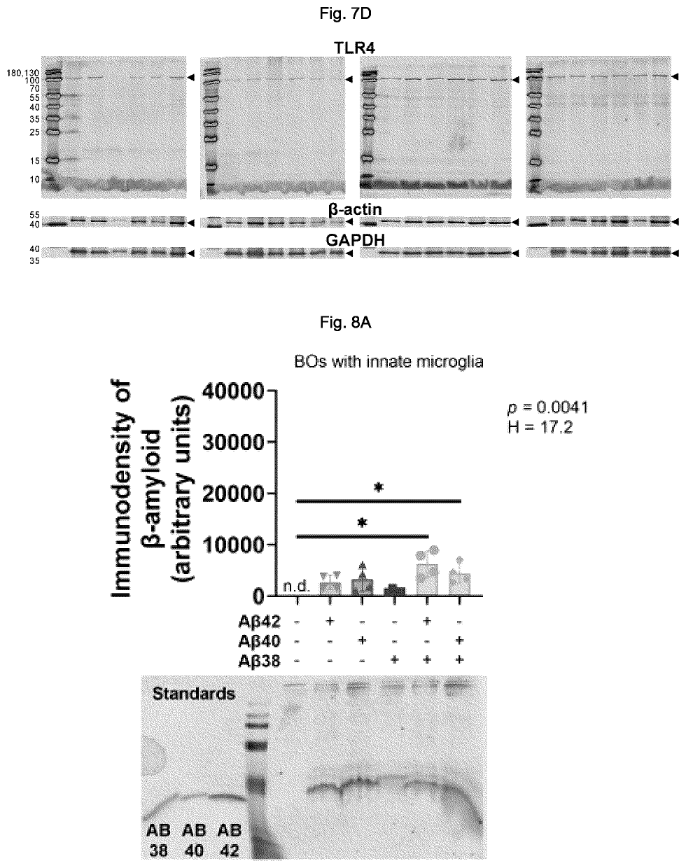

FIGS. 7A-D demonstrate the protein banding patterns between adult human brain tissue and adult mouse brain tissue. The different bands on these images could represent different protein isoforms, post-translation modifications, protein splice variants, etc. The differences between human tissue (brain and organoids) and mouse tissue were most striking with respect to NeuN and TMEM119. With respect to GFAP, human tissues appeared to have multiple bands, unlike mouse tissues.

BOs can be grown with consistent protein profiles, and exhibited similar or less variability than post-mortem human brain samples and clonal mouse brains. Unlike mouse brain tissue, BOs expressed proteins at molecular weights and levels similar the human brain.

Various stressors, including the Alzheimer's disease (AD)-related β-amyloid peptide, are known to activate microglia. A comparison of the response of a) brain organoids in which microglia were integrated by co-culture (BORG co-cultures), or b) brain organoids in which microglia were generated innately using specific molecular cocktails (BORG innate cultures) to acute treatments of β-amyloid peptides of varying lengths was performed. Levels of ‘ionized calcium-binding adapter molecule 1’ (IBA1) were used as a marker of microglial activation. It was demonstrated herein that the IBA1 levels of BORG co-cultures dramatically increased in response to β-amyloid 1-42 and 1-42 plus 1-38, whereas the IBA1 levels of BORG innate cultures remained unaffected after a 24 h exposure.

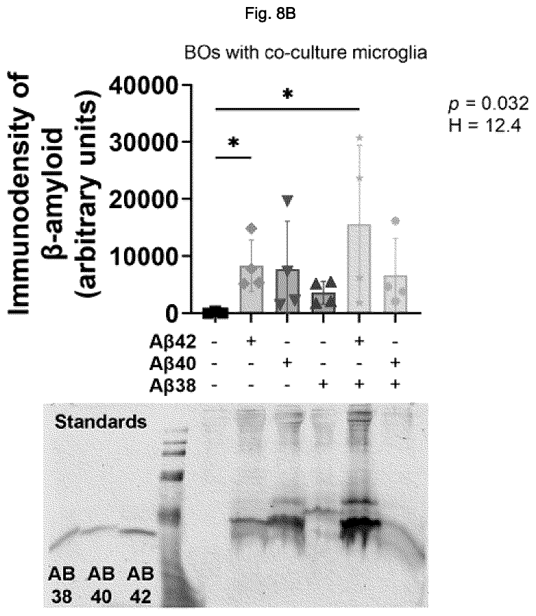

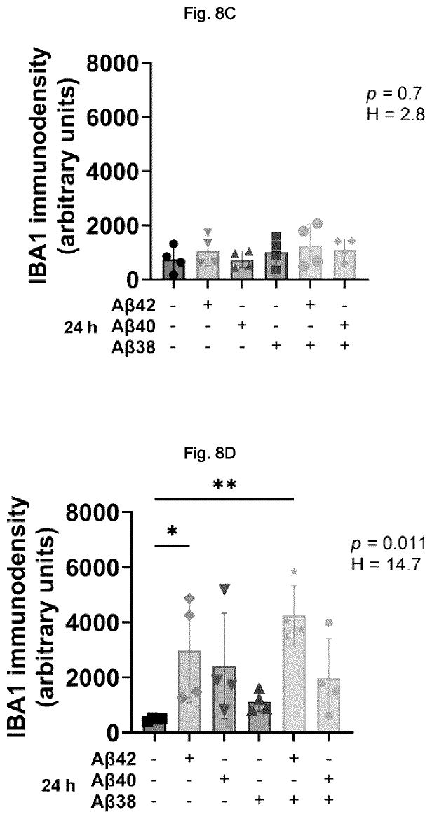

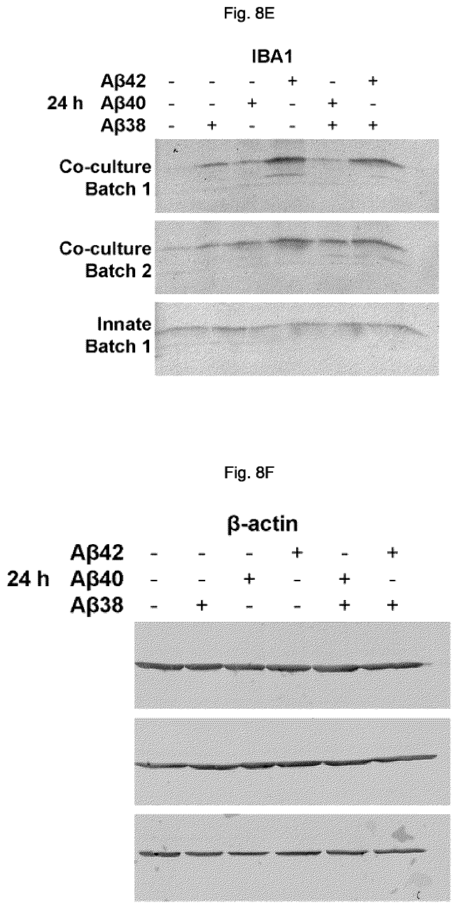

FIGS. 8A-F demonstrate that microglia grown innately in brain organoids responded to externally-administered amyloid-beta differently than microglia that were added to brain organoids by co-culture, as measured by the levels of the microglial protein IBA1. Other protocols predominately rely on microglia being co-cultured with organoids to introduce this cell type.

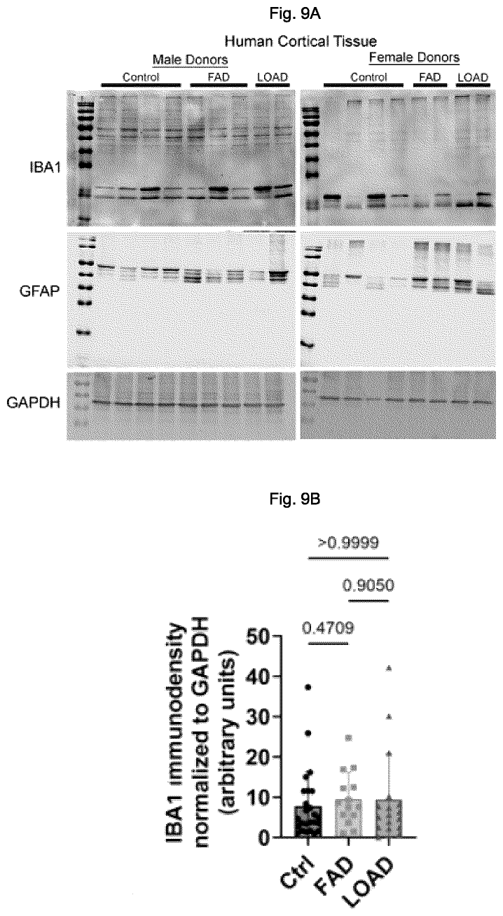

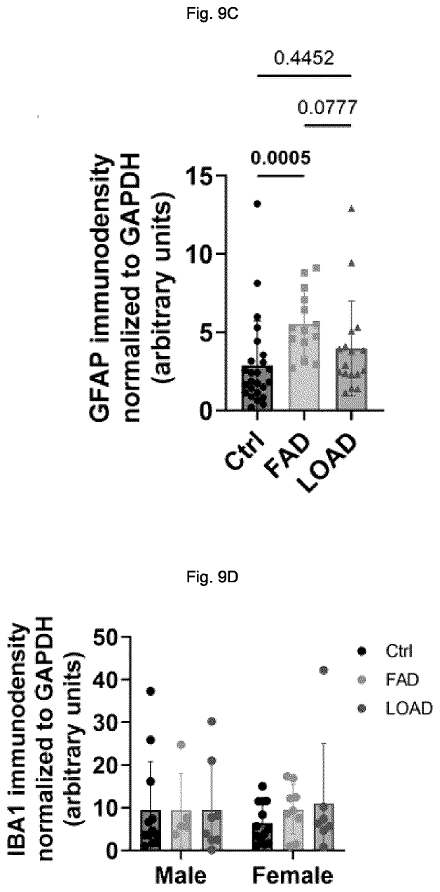

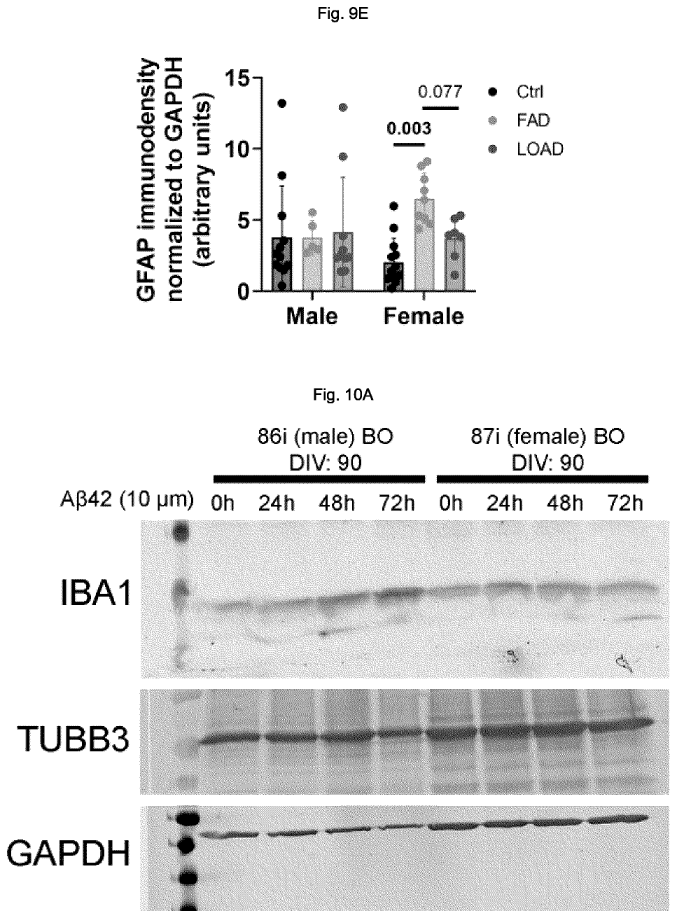

It is widely cited in the literature that IBA1 increases in animal models of Alzheimer's disease, but it is less clear whether this occurs in the brains of patients with Alzheimer's disease. As is demonstrated in FIGS. 9A-E, IBA1 levels did not underscore any subtype of Alzheimer's disease (familial, FAD; late-onset Alzheimer's disease, LOAD). This suggests that increasing levels of beta-amyloid in the brain do not trigger an increase in IBA1, and therefore increases in IBA1 in response to beta-amyloid are not a pathophysiological human response. This indicates that the presently described brain organoid model with innate microglia in FIGS. 8A-F is closer to a human-like response than the brain organoids generated with microglia using other protocols (i.e., co-culture).

FIGS. 10A-C demonstrate that beta-amyloid externally administered to brain organoids with innate microglia had a transient effect in female brain organoids (see 48 h timepoint), and a progressive increasing response in male brain organoids (see 72 h timepoint). This indicates that brain organoids do have some capacity to respond to beta-amyloids, but it is nowhere near as strong as the non-pathophysiological response exhibited by brain organoids generated with microglia using other protocols (i.e., co-culture).

As mentioned above, FIGS. 10A-C show IBA1 levels of innate microglia had their IBA1 levels increase in response to externally-administered amyloid-beta, but at a later timepoint (48-72 h). This increase at 48-72 h was not as pronounced as the response with co-culture microglia. Interestingly, in post-mortem human brain tissue of Alzheimer's disease patients, these human brain tissues did not exhibit increased levels of IBA1, suggesting that this is predominately a phenotype only seen in an animal model of Alzheimer's disease. As the post-mortem human brain tissue-which have a high burden of amyloid-beta-did not exhibit high levels of IBA1, this suggests that IBA1 levels should not dramatically increase in response to any amyloid-beta treatment. Thus, the data presented herein suggests that innate microglia exhibit more physiological (and translationally-relevant) responses, which is important for disease modelling and drug screening.

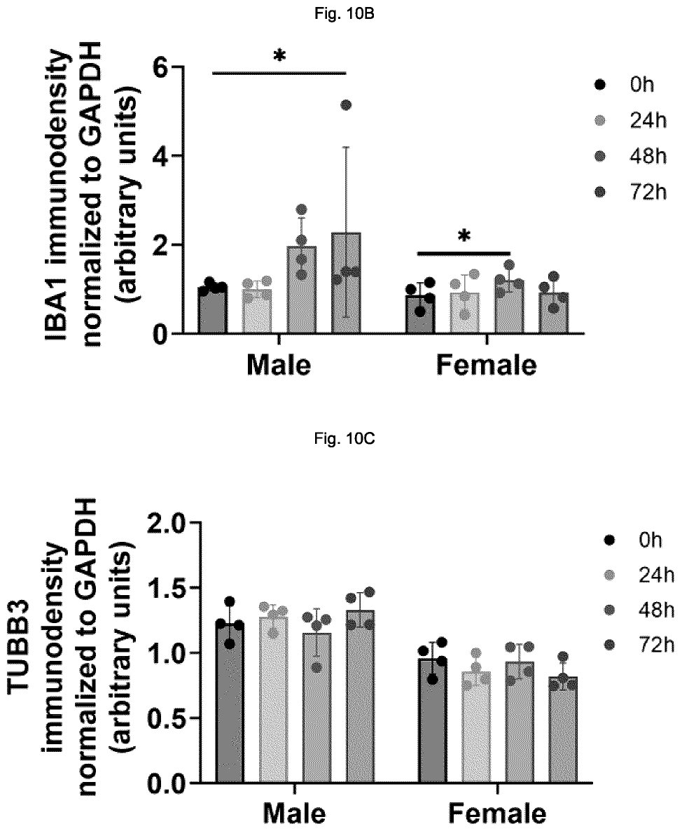

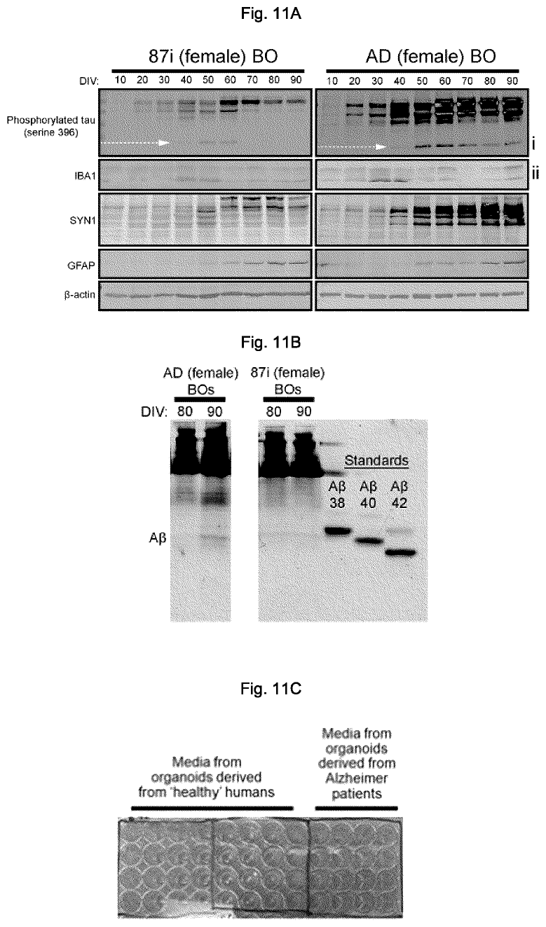

FIGS. 11A-C compare the differences between brain organoids generated from induced pluripotent stem cells (iPSCs) derived from a female Alzheimer's disease patient (AD brain organoid), and brain organoids generated from iPSCs from a neurocognitively healthy female donor (female BO). This female Alzheimer's disease patient was diagnosed with late-onset Alzheimer's disease, and this patient did not carry any recognized genetic risk factors for Alzheimer's disease. Thus, this patient had what is termed ‘sporadic’ Alzheimer's disease and accounts for roughly 90% of the cases of Alzheimer's disease globally.

All relevant published studies used iPSCs with genetic risk factors of Alzheimer's disease (i.e., those for early-onset Alzheimer's disease, such as presenilin 1 and amyloid protein precursor mutations, or late-onset Alzheimer's disease, such as apolipoprotein E ε4/ε4). Therefore, this is believed to be the first dataset to indicate brain organoids can be used for investigating the pathophysiology of Alzheimer's disease, including the subtypes such as familial Alzheimer's disease, sporadic Alzheimer's disease, early-onset Alzheimer's disease and late-onset Alzheimer's disease. Additionally, the brain organoids generated from the Alzheimer's disease patient exhibited the same pathophysiology as the corresponding human brain tissue would, indicating that brain organoids may be able to confirm a diagnosis of Alzheimer's disease, and possibly even predict its onset. It is strongly believed that the brain organoids generated according to the methods described herein can model sporadic Alzheimer's disease due to the presence of microglia in the present model, as microglia are widely implicated in the mechanisms underlying symptom onset.

In FIG. 11A, it was shown that the AD brain organoids expressed a 24 kilodalton protein detected by phosphorylated tau (serine 396) antibodies (marked by a white arrow in the blot labelled “i”). This 24 kilodalton protein has been implicated in both humans with Alzheimer's disease and in mouse models of Alzheimer's disease. In comparison, the female BO only expressed this protein transiently and at much lower levels. Preceding the appearance of the 24 kilodalton tau protein was an increase in the expression of IBA1 (see the blot labelled “ii”). Again, an increase in IBA1 has been described to precede the appearance of pathological proteins (i.e., amyloid-betas and phosphorylated tau) in human Alzheimer's disease.

In FIG. 11B, it was also shown that the female AD brain organoids also had much higher levels of amyloid-beta peptides than healthy female brain organoids. In FIG. 11C it was also shown that AD brain organoids exhibited the hypermetabolic state of Alzheimer's disease after 50 days in vitro (DIV), as measured by a striking reduction in pH (i.e., when in colour, FIG. 11C shows yellowing media which indicates reduction in pH). This hypermetabolic state has often been attributed to the ‘mild cognitive impairment’ stage of human Alzheimer's disease. In summary, markers of Alzheimer's disease that the brain organoid platform presently described herein can model were demonstrated, and the data described herein lays the promising foundation for use of the described platform for drug screening and as a diagnostic/predictive tool.

### Example 3

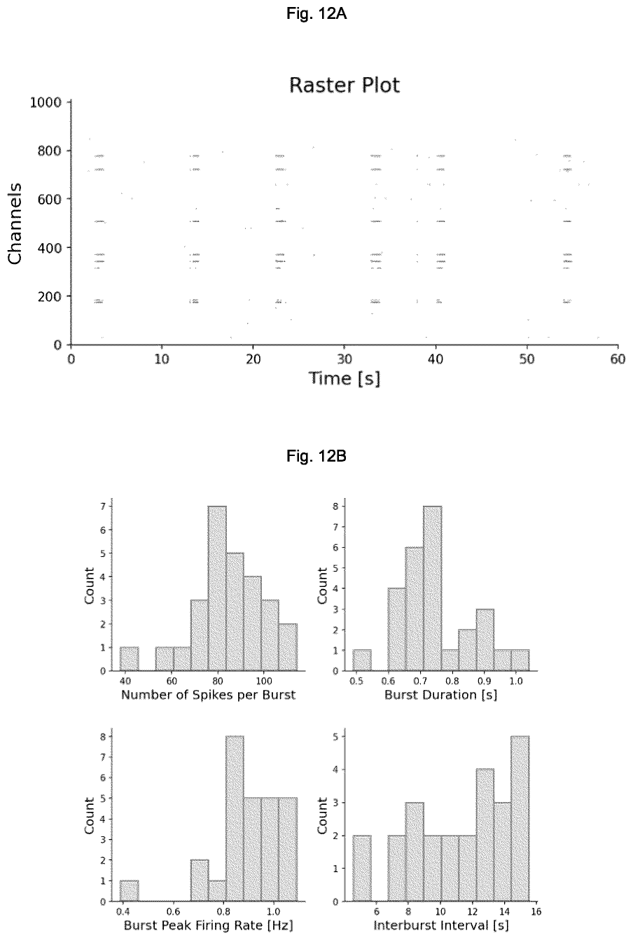

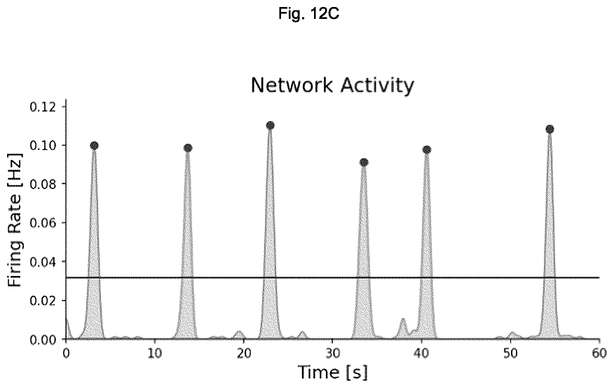

Multielectrode array experiments were performed to test whether the brain organoids (BOs) described herein exhibited neural activity in vitro at day 90. Two weeks after plating the organoids, spontaneous and synchronized neural network electrical activity was detected (FIGS. 12A-C).

Some studies generating BOs without microglia, using methods other than the ones described herein, observe synchronized activity between five to six months in culture (Fair et al., 2020; Sharf et al., 2022), or not at all (Popova et al., 2021). Popova et al. (2021) compared their results to BOs with microglia being incorporated at 4-months through a co-culture method (i.e., not the innate method described herein) and could detect spontaneous synchronicity at 5 months of culture. This suggests the microglia-like cells in the BOs described herein may be underpinning the ability to detect synchronized neural network activity in the brain organoids described herein at three months in culture (i.e., 90 days after embryoid body formation). Halving the amount of time required to get brain-like spontaneous burst activity is beneficial for any application of brain organoids, as it greatly reduces the cost of the platform and mitigates risks (i.e., contamination, network activity never developing due to some unexpected issue early on in organoid development).

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent, or patent application was specifically and individually indicated to be incorporated by reference in its entirety. Specifically, the sequences associated with each accession numbers provided herein including for example accession numbers for proteins and/or nucleic acid provided in the Tables or elsewhere, are incorporated by reference in its entirely.

The scope of the claims should not be limited by the preferred embodiments and examples but should be given the broadest interpretation consistent with the description as a whole.

### REFERENCES

1. Abcam, n.d. Anti-PSD95 antibody (ab12093), https://www.abcam.com/PSD95-antibody-ab12093.html.
2. Cowan, M., Petri, W. A., 2018. Microglia: immune regulators of neurodevelopment. Front Immunol.
3. Cristovão, G., Pinto, M. J., Cunha, R. A., Almeida, R. D., Gomes, C. A., 2014. Activation of microglia bolsters synapse formation. Front Cell Neurosci 8.
4. Curran, O. E., Qiu, Z., Smith, C., Grant, S. G. N., 2021. A single-synapse resolution survey of PSD95-positive synapses in twenty human brain regions. Eur J Neurosci 54, 6864-6881.
5. Fair, S. R., Julian, D., Hartlaub, A. M., Pusuluri, S. T., Malik, G., Summerfied, T. L., Zhao, G., Hester, A. B., Ackerman I V, W. E., Hollingsworth, E. W., Ali, M., McElroy, C. A., Buhimschi, I. A., Imitola, J., Maitre, N. L., Bedrosian, T. A., Hester, M. E., 2020. Electrophysiological maturation of cerebral organoids correlates with dynamic morphological and cellular development. Stem Cell Reports 15, 855-868.
6. Droguerre, M., Tsurugizawa, T., Duchêne, A., Portal, B., Guiard, B. P., Déglon, N., Rouach, N., Hamon, M., Mouthon, F., Ciobanu, L., Charveriat, M., 2019. A new tool for in vivo study of astrocyte connexin 43 in brain. Sci Rep 9, 18292.
7. Gumbs, S. B. H., Berlekom, A. B. Van, Kübler, R., Schipper, P. J., Gharu, L., Boks, M. P., Ormel, P. R., Wensing, A. M. J., Witte, L. D. De, Nijhuis, M., 2022. Characterization of HIV-1 infection in microglia-containing human cerebral organoids. Viruses 14, 829.
8. Konishi, H., Okamoto, T., Hara, Y., Komine, O., Tamada, H., Maeda, M., Osako, F., Kobayashi, M., Nishiyama, A., Kataoka, Y., Takai, T., Udagawa, N., Jung, S., Ozato, K., Tamura, T., Tsuda, M., Yamanaka, K., Ogi, T., Sato, K., Kiyama, H., 2020. Astrocytic phagocytosis is a compensatory mechanism for microglial dysfunction. EMBO J 39, e104464.
9. Lancaster, M. A., Knoblich, J. A., 2014. Generation of cerebral organoids from human pluripotent stem cells. Nat Protoc 9, 2329-2340.
10. Lukens, J. R., Eyo, U. B., 2022. Microglia and neurodevelopmental disorders. Annu Rev Neurosci.
11. Ormel, P. R., Vieira de Sá, R., van Bodegraven, E. J., Karst, H., Harschnitz, O., Sneeboer, M. A. M., Johansen, L. E., van Dijk, R. E., Scheefhals, N., Berdenis van Berlekom, A., Ribes Martinez, E., Kling, S., MacGillavry, H. D., van den Berg, L. H., Kahn, R. S., Hol, E. M., de Witte, L. D., Pasterkamp, R. J., 2018. Microglia innately develop within cerebral organoids. Nat Commun 9, 4167.
12. Popova, G., Soliman, S. S., Kim, C. N., Keefe, M. G., Hennick, K. M., Jain, S., Li, T., Tejera, D., Shin, D., Chhun, B. B., McGinnis, C. S., Speir, M., Gartner, Z. J., Mehta, S. B., Haeussler, M., Hengen, K. B., Ransohoff, R. R., Piao, X., Nowakowski, T. J., 2021. Human microglia states are conserved across experimental models and regulate neural stem cell responses in chimeric organoids. Cell Stem Cell 28, 2153-2166.e6.
13. Sharf, T., van der Molen, T., Glasauer, S. M. K., Guzman, E., Buccino, A. P., Luna, G., Cheng, Z., Audouard, M., Ranasinghe, K. G., Kudo, K., Nagarajan, S. S., Tovar, K. R., Petzold, L. R., Hierlemann, A., Hansma, P. K., Kosik, K. S., 2022. Functional neuronal circuitry and oscillatory dynamics in human brain organoids. Nat Commun 13, 4403.
14. Sivitilli, A. A., Gosio, J. T., Ghoshal, B., Evstratova, A., Trcka, D., Ghiasi, P., Hernandez, J. J., Beaulieu, J. M., Wrana, J. L., Attisano, L., 2020. Robust production of uniform human cerebral organoids from pluripotent stem cells. Life Sci Alliance 3, e202000707.
15. Tanaka, Y., Cakir, B., Xiang, Y., Sullivan, G. J., Park, I. H., 2020. Synthetic analyses of single-cell transcriptomes from multiple brain organoids and fetal brain. Cell Rep 30, 1682-1689.e3.
16. Lier, Julia et al. “Beyond Activation: Characterizing Microglial Functional Phenotypes.” Cells vol. 10, 9 2236. 28 Aug. 2021, doi: 10.3390/cells10092236.
17. Ohsawa, Keiko et al. “Microglia/macrophage-specific protein Iba1 binds to fimbrin and enhances its actin-bundling activity.” Journal of neurochemistry vol. 88, 4 (2004): 844-56. doi: 10.1046/j. 1471-4159.2003.02213.
18. Yang, Ben et al. “Transmembrane protein TMEM119 facilitates the stemness of breast cancer cells by activating Wnt/β-catenin pathway.” Bioengineered vol. 12, 1 (2021): 4856-4867. doi: 10.1080/21655979.2021.1960464.
16. Gosselin, David et al. “An environment-dependent transcriptional network specifies human microglia identity.” Science (New York, N.Y.) vol. 356, 6344 (2017): eaal3222. doi: 10.1126/science.aal3222.

1. A method of generating at least one brain organoid-sufficient embryoid body, the method comprising incubating a population of induced pluripotent stem cells (iPSCs) in an embryoid body formation medium (EB FM), the EB FM comprising:
a cell culture medium, optionally Dulbecco's modified Eagle medium (DMEM)
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin;
about 5 ng/ml to about 30 ng/ml of sodium selenite; and
about 1 ng/ml to about 200 ng/ml, optionally about 10 ng/ml to about 25 ng/ml, of thermostable fibroblast growth factor 2 (FGF2).

2. The method of claim 1, wherein the concentration of the glutamine in the EB FM is about 2 mM, the concentration of the recombinant human insulin in the EB FM is about 20 μg/mL, the concentration of the recombinant human transferrin in the EB FM is about 20 μg/ml, the concentration of the sodium selenite in the EB FM is about 20 ng/ml, and/or the concentration of the thermostable FGF2 in the EB FM is about 4 ng/ml or about 50 ng/ml.

3. The method of claim 1, wherein the EB FM further comprises:
about 1 μg/ml to about 1 mg/ml L-ascorbic acid (LAA), optionally about 0.2 mg/ml;
about 5 mM to about 45 mM of sodium bicarbonate, optionally about 14.7 mM;
about 0.1 ng/ml to about 20 ng/ml of transforming growth factor beta-3 (TGF-β3) or about 1 ng/ml to about 200 ng/ml transforming growth factor beta-1 (TGF-β1), optionally wherein the concentration of the TGF-β3 in the EB FM is about 0.1 ng/ml; and/or
about 0.1 ng/ml to about 100 ng/ml neuregulin 1 (NRG1), optionally about 0.1 ng/ml.

4. (canceled)

5. The method of claim 1, wherein the population of iPSCs is incubated in the EB FM for at least about 24 hours, optionally for at least about 24 hours and up to about 144 hours, optionally for about 96 hours.

6. (canceled)

7. (canceled)

8. The method of claim 1, wherein the method further comprises incubating the iPSCs in about 0.05 mM to about 5 mM ethylenediaminetetraacetic acid (EDTA) prior to incubation with the EB FM, optionally wherein the concentration of EDTA is 0.5 mM, optionally wherein the iPSCs are incubated in EDTA for at least about 2 minutes and up to about 10 minutes, optionally for about 4 minutes.

9. (canceled)

10. (canceled)

11. A method of repairing at least one non-viable embryoid body, the method comprising:
obtaining a population of embryoid bodies (EBs) comprising at least one non-viable embryoid body, and
incubating the population of embryoid bodies, optionally for at least about 24 hours, with an embryoid body formation medium (EB FM) comprising:
a cell culture medium, optionally Dulbecco's modified Eagle medium (DMEM);
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin; and
about 5 ng/ml to about 30 ng/ml of sodium selenite.

12. The method of claim 11, wherein the EB FM further comprises about 1 ng/ml to about 200 ng/ml thermostable FGF2, optionally about 10 ng/ml to about 25 ng/ml, optionally about 4 ng/ml or about 50 ng/ml.

13. (canceled)

14. The method of claim 11, wherein the concentration of the glutamine in the EB FM is about 2 mM, the concentration of the recombinant human insulin in the EB FM is about 20 μg/mL, the concentration of the recombinant human transferrin in the EB FM is about 20 μg/m, and/or the concentration of the sodium selenite in the EB FM is about 20 ng/ml.

15. The method of claim 11, wherein the EB FM further comprises:
about 1 μg/ml to about 1 mg/ml L-ascorbic acid (LAA), optionally about 0.2 mg/ml;
about 5 mM to about 45 mM of sodium bicarbonate, optionally about 14.7 mM;
about 0.1 ng/ml to about 20 ng/ml of transforming growth factor beta-3 (TGF-β3) or about 1 ng/ml to about 200 ng/ml transforming growth factor beta-1 (TGF-β1), optionally wherein the concentration of the TGF-β3 in the EB FM is about 0.1 ng/ml; and/or
about 0.1 ng/ml to about 100 ng/ml neuregulin 1 (NRG1), optionally about 0.1 ng/ml.

16. (canceled)

17. (canceled)

18. A method of generating at least one brain organoid, the method comprising
a. generating at least one brain organoid-sufficient embryoid body using the method of claim 1 and/or generating at least one repaired brain organoid-sufficient embryoid body using a method of repairing at least one non-viable embryoid body;
b. transferring the at least one brain organoid-sufficient embryoid body generated in step a) from the EB FM used in step a) to a neural induction medium without transferring any of the EB FM into the neural induction medium and incubating the at least one brain organoid-sufficient embryoid body in the neural induction medium for at least about 24 hours and up to about 96 hours, optionally for about 48 hours;
c. replacing the neural induction medium with expansion medium having a temperature of at least about 0° C. and up to about 12° C., optionally about 4° C. to about 6° C.; and
d. replacing the expansion medium with maturation medium after at least about 48 hours up to about 96 hours, optionally at least 72 hours,
wherein the method of repairing at least one non-viable embryoid body comprises
obtaining a population of embryoid bodies (EBs) comprising at least one non-viable embryoid body, and
incubating the population of embryoid bodies with an embryoid body formation medium (EB FM) comprising:
a cell culture medium, optionally Dulbecco's modified Eagle medium (DMEM):
about 1 μM to about 100 mM glutamine, optionally about 0.1 mM to about 10 mM L-alanyl-L-glutamine dipeptide;
about 5 μg/ml to about 30 μg/ml of recombinant human insulin;
about 5 μg/ml to about 30 μg/ml of recombinant human transferrin; and
about 5 ng/ml to about 30 ng/ml of sodium selenite.

19. The method of claim 18, wherein the at least one brain organoid comprises astrocytes, neurons, oligodendrocytes, and/or microglia, optionally wherein the at least one brain organoid comprises microglia and astrocytes, optionally wherein the at least one brain organoid comprises microglia.

20. (canceled)

21. (canceled)

22. The method of claim 18, wherein the neural induction medium comprises
DMEM-F12 with HEPES;
20 μg/ml of recombinant human transferrin;
20 μg/ml of recombinant human insulin;
20 ng/ml of sodium selenite;
2 mM of L-alanyl-L-glutamine dipeptide;
0.2 mg/ml of LAA; and
1 μg/ml of heparin.

23. The method of claim 18, wherein the expansion medium comprises
1:1 mixture of DMEM-F12 with HEPES:Neurobasal medium;
20 μg/μl of recombinant human transferrin;
20 μg/μl of recombinant human insulin;
20 ng/ml of sodium selenite;
2 mM of L-alanyl-L-glutamine dipeptide;
0.2 mg/ml of LAA;
2×S15 without vitamin A supplement; and
2% v/v of undiluted hESC-qualified Matrigel matrix.

24. The method of claim 18, wherein the maturation medium comprises
1:1 mixture of DMEM-F12 with HEPES and Neurobasal medium;
20 ng/ml of sodium selenite;
2 mM of L-alanyl-L-glutamine dipeptide;
0.2 mg/ml of LAA; and
2×S15 with vitamin A supplement.

25. A brain organoid generated using the methods of claim 18.

26. The brain organoid of claim 25, wherein neural network electrical activity is detectable in the brain organoid at least 90 days after the generation of the at least one brain organoid-sufficient embryoid body and/or wherein the brain organoid is generated from iPSCs from a patient having Alzheimer's disease, optionally wherein the Alzheimer's disease is sporadic Alzheimer's disease.

27. (canceled)

28. (canceled)

29. A brain organoid-sufficient embryoid body repaired using the methods of claim 11.

30. A method of diagnosing or predicting onset of Alzheimer's disease in a patient suspected of having or developing Alzheimer's disease, the method comprising
a) collecting a sample from the patient,
b) isolating cells from the sample,
c) reprogramming the cells into induced pluripotent stem cells (iPSCs),
d) using the iPSCs to generate a brain organoid using the method of claim 18, and
e) detecting the presence of markers associated with Alzheimer's disease in the brain organoid.

31. The method of claim 30, wherein the markers associated with Alzheimer's disease comprise the presence of a phosphorylated tau protein in the brain organoid, an increase in the levels of ionized calcium-binding adapter molecule 1 (IBA1) as compared to a control, an increase in the levels of amyloid-beta peptides as compared to a control, and/or a reduction in pH as compared to a control.

32. The method of claim 30, wherein the Alzheimer's disease is sporadic Alzheimer's Disease.


SEQUENCE LISTING (3 sequences)

SEQ ID NO: 1 - protein, 155 aa, synthetic construct
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALNRTGQYKLGSKTGPGQKAILFLPMSAKS

SEQ ID NO: 2 - protein, 155 aa, synthetic construct
MAAGSITTLPALPEDGGSGAFPPGHFKDPKLLYCKNGGFFLRIHPDGRVDGTRDKSDPFIKLQLQAEERGVVSIKGVCANRYLAMKEDGRLYAIKNVTDECFFFERLEENNYNTYRSRKYPSWYVALKRTGQYKLGSKTGPGQKAILFLPMSAKS

SEQ ID NO: 3 - protein, 155 aa, synthetic construct [sequence omitted]